(19) United States
(12) Patent Application Publication
(10) Pub. No.: US 2023/0357870 A1
(43) Pub. Date: Nov. 9, 2023

(54) CORONAVIRUS REPLICONS FOR ANTIVIRAL SCREENING AND TESTING

(71) Applicant: MERCK SHARP & DOHME LLC, Rahway, NJ (US)

(72) Inventors: Xi HE, Philadelphia, PA (US); Shuo QUAN, Cranbury, NJ (US); Dai WANG, Blue Bell, PA (US); Min XU, Ambler, PA (US)

(73) Assignee: MERCK SHARP & DOHME LLC, Rahway, NJ (US)

(21) Appl. No.: 18/246,408

(22) PCT Filed: Sep. 23, 2021
(86) PCT No.: PCT/US2021/051626

§ 371 Date: Mar. 23, 2023

Related U.S. Application Data
(60) Provisional application No. 63/083,852, filed on Sep. 25, 2020.

(51) Int. Cl. C12Q 1/6897; C12N 15/86

(52) U.S. Cl. CPC C12Q 1/6897; C12N 15/86; C12Q 2600/136; C12Q 2600/158; C12N 2770/20043

(57) ABSTRACT

This application provides materials and methods related to replication competent, noninfectious coronavirus reporter replicons, such as for SARS-CoV-2, MERS, or SARS-CoV-1 comprising at least one coronavirus gene and at least one reporter gene. The application also provides methods for assaying candidate agents for inhibition of coronavirus viral replication.

### CROSS-REFERENCE TO RELATED APPLICATIONS

This application claims the benefit of U.S. Provisional Patent Application Ser. No. 63/083,852 filed Sep. 25, 2020, the entire contents of which are incorporated by reference herein.

### REFERENCE TO SEQUENCE LISTING SUBMITTED ELECTRONICALLY

The instant application contains a Sequence Listing which has been submitted electronically in ASCII format and is hereby incorporated by reference in its entirety. The ASCII copy, created on Sep. 21, 2021, is named 25110WOPCT-SEQTXT-21SEP2021.txt and is 662,700 bytes in size.

### FIELD

Noninfectious reporter replicons for coronavirus.

### BACKGROUND OF THE INVENTION

The current pandemic of coronavirus disease 2019 (COVID-19) caused by the newly emerged coronavirus, severe acute respiratory syndrome coronavirus 2 (SARS-CoV-2) has led to nearly 206 million infections and over 4.3 million deaths as of Aug. 12, 2021. In response to this public health emergency, an unprecedented swift and concerted effort has been launched globally to identify safe and effective therapeutics and vaccines against the rapidly spreading virus. Presently, about 2000 SARS-CoV-2 investigational programs and clinical trials have been registered (Thorlund, Kristian et al. “A real-time dashboard of clinical trials for COVID-19.” The Lancet. Digital health vol. 2, 6 (2020): e286-e287. doi:10.1016/S2589-7500(20)30086-8). However, most of the treatment trials focus on repurposing a limited number of existing antivirals approved for other indications, such as lopinavir/ritonavir or remdesivir (GS-5734™; VEKLURY®). Although compounds directly targeting SARS-CoV-2 are urgently needed, development of such a compound typically requires a decade of research. One of the major bottlenecks is that assays used to screen and test SARS-CoV-2 compounds involving the live virus must be performed in BSL3 laboratories, which are not readily available to most researchers.

### SUMMARY OF THE INVENTION

The present disclosure provides materials and methods relating to a replication competent, but noninfectious coronavirus reporter replicons, including SARS-CoV-2, SARS-CoV-1 and MERS, for evaluating the potencies of RNA-dependent RNA polymerase inhibitors and/or protease inhibitors.

In accordance with the description, a coronavirus reporter replicon comprising: one or more coronavirus genes encoding one or more coronavirus proteins for coronavirus viral RNA replication; and at least one reporter gene; wherein the coronavirus reporter replicon does not comprise at least one of a functional coronavirus spike ORF, envelope ORF, or membrane ORF, and wherein the coronavirus reporter replicon is replication competent. In some embodiments, the one or more coronavirus genes comprise or are transcribed into a nucleic acid sequence that is a target for a candidate antiviral agent; or the one or more coronavirus genes encode a protein target for a candidate antiviral agent, and wherein the coronavirus reporter replicon is replication competent.

In some embodiments, the one or more coronavirus genes are SARS-CoV-2 genes chosen from ORF1a (positions 266-13,480 of SEQ ID NO: 1), ORF1ab (positions 266-21,555 of SEQ ID NO: 1), ORF3a (positions 25,393-26,220 of SEQ ID NO: 1), ORF6 (positions 27,202-27,387 of SEQ ID NO: 1), ORF7a (positions 27,394-27,759 of SEQ ID NO: 1), ORF7b (positions 27,756-27,887 of SEQ ID NO: 1), ORF8 (positions 27,894-28,259 of SEQ ID NO: 1), and ORF10 (positions 29,558-29,674 of SEQ ID NO: 1), and sequences are at least 90%, 91%, 92%, 93%, 94%, 95%, 96%, 97%, 98%, or 99% homologous to those genes. In some embodiments, the one or more SARS-CoV-2 genes comprise ORF1ab (positions 266-21,555 of SEQ ID NO: 1), ORF3a (positions 25,393-26,220 of SEQ ID NO: 1), ORF6 (positions 27,202-27,387 of SEQ ID NO: 1), ORF7a/b (positions 27,394-27,887 of SEQ ID NO: 1), ORF8 (positions 27,894-28,259 of SEQ ID NO: 1), and ORF10 (positions 29,558-29,674 of SEQ ID NO: 1), and sequences that are at least 90%, 91%, 92%, 93%, 94%, 95%, 96%, 97%, 98%, or 99% homologous to those genes. In some embodiments, the one or more SARS-CoV-2 genes comprise ORF1ab (positions 266-21,555 of SEQ ID NO: 1), ORF3a (positions 25,393-26,220 of SEQ ID NO: 1), ORF6 (positions 27,202-27,387 of SEQ ID NO: 1), ORF7a/b (positions 27,394-27,887 of SEQ ID NO: 1), ORF8 (positions 27,894-28,259 of SEQ ID NO: 1), and ORF10 (positions 29,558-29,674 of SEQ ID NO: 1).

In some embodiments of the coronavirus reporter replicon disclosed herein, the one or more coronavirus genes are Middle East Respiratory Syndrome (MERS) genes chosen from ORF1a (positions 279-21,514 of SEQ ID NO: 27), ORF3 (positions 25,532-25,843 of SEQ ID NO: 27), ORF4a (positions 25,852-26,181 of SEQ ID NO: 27), ORF4b (positions 26,093-26,833 of SEQ ID NO: 27), ORF5 (positions 26,840-27-514 of SEQ ID NO: 27), and ORF8b (positions 28,762-29,100 of SEQ ID NO: 27). In some embodiments, the one or more MERS genes comprise ORF1a (positions 279-21,514 of SEQ ID NO: 27), ORF3 (positions 25,532-25,843 of SEQ ID NO: 27), ORF4a (positions 25,852-26,181 of SEQ ID NO: 27), ORF4b (positions 26,093-26,833 of SEQ ID NO: 27), ORF5 (positions 26,840-27-514 of SEQ ID NO: 27), and ORF8b (positions 28,762-29,100 of SEQ ID NO: 27).

In some embodiments of the coronavirus reporter replicon disclosed herein, the one or more coronavirus genes are SARS-CoV-1 genes chosen from ORF1ab (positions 250-21,467 of SEQ ID NO: 31), ORF3a (positions 25,253-26,077 of SEQ ID NO: 31), ORF6 (positions 27,059-27,250 of SEQ ID NO: 31), ORF7a (positions 27,258-27,626 of SEQ ID NO: 32), ORF7b (positions 27,623-27,757 of SEQ ID NO: 31), ORF8a (positions 27,764-27,883 of SEQ ID NO: 31), and ORF8b (positions 27,849-28,103 of SEQ ID NO: 31). In some embodiments, the one or more SARS-CoV-1 genes comprise ORF1ab (positions 250-21,467 of SEQ ID NO: 31), ORF3a (positions 25,253-26,077 of SEQ ID NO: 31), ORF6 (positions 27,059-27,250 of SEQ ID NO: 31), ORF7a (positions 27,258-27,626 of SEQ ID NO: 32), ORF7b (positions 27,623-27,757 of SEQ ID NO: 31), ORF8a (positions 27,764-27,883 of SEQ ID NO: 31), and ORF8b (positions 27,849-28,103 of SEQ ID NO: 31).

In some embodiments, the coronavirus reporter replicon does not comprise a functional coronavirus spike ORF. In some embodiments, the coronavirus reporter replicon does not comprise a functional coronavirus envelope ORF. In some embodiments, the coronavirus reporter replicon does not comprise a functional coronavirus membrane ORF. In some embodiments, the coronavirus reporter replicon does not comprise a functional coronavirus spike ORF, envelope ORF, or membrane ORF. In some embodiments, the coronavirus reporter replicon does not comprise at least one of a functional spike ORF, envelope ORF, or membrane ORF.

In some embodiments, one or more of the functional spike ORF, the envelope ORF, and the membrane ORF is replaced with the reporter gene. In some embodiments, one or more of the functional spike ORF, the envelope ORF, and the membrane ORF is replaced with a selection gene.

In some embodiments, at least one of the coronavirus genes encodes RNA-dependent RNA polymerase (RdRp). In some embodiments, the RdRp comprises at least one mutation compared to SEQ ID NO: 9. In some embodiments, the RdRp sequence is at least 90%, 91%, 92%, 93%, 94%, 95%, 96%, 97%, 98%, or 99% homologous to SEQ ID NO: 9.

In some embodiments, at least one of the coronavirus genes is the protease Mpro.

In some embodiments, the at least one reporter gene comprises a bioluminescent reporter gene, a fluorescent protein gene, and/or an enzymatic reporter gene. In some embodiments, the at least one reporter gene comprises a gene for firefly luciferase, Renilla luciferase, nanoluciferase, Gaussia luciferase, GFP, eGFP, RFP, mCherry, DsRed, β-lactamase, β-galactosidase, and/or secreted alkaline phosphatase. In some embodiments, the at least one reporter gene comprises firefly luciferase. In some embodiments, the at least one reporter gene comprises GFP. In some embodiments, the at least one reporter gene comprises a fusion gene of firefly luciferase and GFP.

In some embodiments, the coronavirus reporter replicon further comprises a selection gene. In some embodiments, the selection gene comprises an antibiotic resistance gene. In some embodiments, the selection gene imparts resistance to neomycin, puromycin, and/or blasticidin. In some embodiments, the selection gene comprises neomycin phosphotransferase, puromycin N-acetyltransferase, or blasticidin resistance gene.

In some embodiments, the coronavirus reporter replicon further comprises a nucleocapsid ORF. In some embodiments, the coronavirus reporter replicon comprises replicase complex ORF1ab, a reporter gene, a selection gene, and a nucleocapsid ORF. In some embodiments, the coronavirus reporter replicon comprises an ORF1a, an ORF1ab, a fusion gene of firefly luciferase and GFP, a neomycin phosphotransferase gene, and a nucleocapsid ORF.

In some embodiments, the coronavirus reporter replicon comprises nucleotides 284-28,234 of SEQ ID NO: 2, nucleotides 284-26,118 of SEQ ID NO: 5, nucleotides 296-28,103 SEQ ID NO: 28, nucleotides 296-28,104 of SEQ ID NO: 29, nucleotides 296-28,034 of SEQ ID NO: 30, or nucleotides 283-27,898 SEQ ID NO: 32, or a sequence that is 90%, 91%, 92%, 93%, 94%, 95%, 96%, 97%, 98%, or 99% homologous to 284-28,234 of SEQ ID NO: 2, nucleotides 284-26,118 of SEQ ID NO: 5, nucleotides 296-28,103 SEQ ID NO: 28, nucleotides 296-28,104 of SEQ ID NO: 29, nucleotides 296-28,034 of SEQ ID NO: 30, or nucleotides 283-27,898 SEQ ID NO: 32. In some embodiments, the coronavirus reporter replicon comprises nucleotides 284-28,234 of SEQ ID NO: 2 or a sequence that is 90%, 91%, 92%, 93%, 94%, 95%, 96%, 97%, 98%, or 99% homologous to nucleotides 19-28,237 of SEQ ID NO: 2. In some embodiments, the coronavirus reporter replicon comprises nucleotides 284-26,118 of SEQ ID NO: 5 or a sequence that is 90%, 91%, 92%, 93%, 94%, 95%, 96%, 97%, 98%, or 99% homologous to nucleotides 20-28,327 of SEQ ID NO: 5. In some embodiments, the coronavirus reporter replicon comprises nucleotides 296-28,103 of SEQ ID NO: 28 or a sequence that is 90%, 91%, 92%, 93%, 94%, 95%, 96%, 97%, 98%, or 99% homologous to nucleotides 296-28,103 of SEQ ID NO: 28. In some embodiments, the coronavirus reporter replicon comprises nucleotides 296-28,104 of SEQ ID NO: 29 or a sequence that is 90%, 91%, 92%, 93%, 94%, 95%, 96%, 97%, 98%, or 99% homologous to nucleotides 296-28,104 of SEQ ID NO: 29. In some embodiments, the coronavirus reporter replicon comprises nucleotides 296-28,034 of SEQ ID NO: 30 or a sequence that is 90%, 91%, 92%, 93%, 94%, 95%, 96%, 97%, 98%, or 99% homologous to nucleotides 296-28,034 of SEQ ID NO: 30. In some embodiments, the coronavirus reporter replicon comprises nucleotides 283-27,898 of SEQ ID NO: 32 or a sequence that is 90%, 91%, 92%, 93%, 94%, 95%, 96%, 97%, 98%, or 99% homologous to nucleotides 283-27,898 of SEQ ID NO: 32.

In some embodiments, the coronavirus reporter replicon comprises a DNA molecule. In some embodiments, the coronavirus reporter replicon comprises an RNA molecule.

The application also provides a vector comprising the sequence encoding the coronavirus reporter replicon of any one of the foregoing embodiments.

In one embodiment, the application provides a vector further comprising at least one promoter; and a terminator cassette. In some embodiments, the vector further comprises a reporter replicon chosen from SEQ ID NO: 2, 5, or 7.

In some embodiments, the at least one promoter comprises a T7 promoter, a T7GG promoter, a T7GGG promoter, an EF-1a promoter, a human pol I promoter, a CMV promoter, SP6, or a CMV/T7 dual promoter. In some embodiments, the promoter comprises a T7 promoter. In some embodiments, the promoter comprises a T7GG promoter. In some embodiments, the promoter comprises a CMV promoter. In some embodiments, the promoter comprises a CMV/T7 dual promoter.

In some embodiments, the terminator cassette comprises a polyA sequence, hepatitis D virus ribozyme sequence, and/or T7 terminator.

In some embodiments, the vector comprises SEQ ID NO: 2, 3, 4, 5, 6, 7, 13, 28, 29, 30, or 32, or a sequence that is 90%, 91%, 92%, 93%, 94%, 95%, 96%, 97%, 98%, or 99% homologous to SEQ ID NO: 2, 3, 4, 5, 6, 7, 13, 28, 29, 30, or 32. In some embodiments, the vector comprises SEQ ID NO: 2, or a sequence that is 90%, 91%, 92%, 93%, 94%, 95%, 96%, 97%, 98%, or 99% homologous to SEQ ID NO: 2. In some embodiments, the vector comprises SEQ ID NO: 3, or a sequence that is 90%, 91%, 92%, 93%, 94%, 95%, 96%, 97%, 98%, or 99% homologous to SEQ ID NO: 3. In some embodiments, the vector comprises SEQ ID NO: 4, or a sequence that is 90%, 91%, 92%, 93%, 94%, 95%, 96%, 97%, 98%, or 99% homologous to SEQ ID NO: 4. In some embodiments, the vector comprises SEQ ID NO: 5, or a sequence that is 90%, 91%, 92%, 93%, 94%, 95%, 96%, 97%, 98%, or 99% homologous to SEQ ID NO: 5. In some embodiments, the vector comprises SEQ ID NO: 6, or a sequence that is 90%, 91%, 92%, 93%, 94%, 95%, 96%, 97%, 98%, or 99% homologous to SEQ ID NO: 6. In some embodiments, the vector comprises SEQ ID NO: 7, or a sequence that is 90%, 91%, 92%, 93%, 94%, 95%, 96%, 97%, 98%, or 99% homologous to SEQ ID NO: 7. In some embodiments, the vector comprises SEQ ID NO: 13, or a sequence that is 90%, 91%, 92%, 93%, 94%, 95%, 96%, 97%, 98%, or 99% homologous to SEQ ID NO: 13. In some embodiments, the vector comprises SEQ ID NO: 28 or a sequence that is 90%, 91%, 92%, 93%, 94%, 95%, 96%, 97%, 98%, or 99% homologous to SEQ ID NO: 28. In some embodiments, the vector comprises SEQ ID NO: 29 or a sequence that is 90%, 91%, 92%, 93%, 94%, 95%, 96%, 97%, 98%, or 99% homologous to SEQ ID NO: 29. In some embodiments, the vector comprises SEQ ID NO: 30 or a sequence that is 90%, 91%, 92%, 93%, 94%, 95%, 96%, 97%, 98%, or 99% homologous to SEQ ID NO: 30. In some embodiments, the vector comprises SEQ ID NO: 32 or a sequence that is 90%, 91%, 92%, 93%, 94%, 95%, 96%, 97%, 98%, or 99% homologous to SEQ ID NO: 32.

The application also provides a cell line expressing the coronavirus reporter replicon of any one of the foregoing embodiments. In some embodiments, a cell line is transfected with a vector of any one of the foregoing embodiments. In some embodiments, a cell line transfected with an in vitro transcribed RNA of the coronavirus reporter replicon of any one of the foregoing embodiments. In some embodiments, the cell line is HEK 293T, Huh7.5, Calu-1, A549, or Vero. In some embodiments, the cell line is not Vero.

The application also provides a method of assaying a candidate agent for inhibition of coronavirus replication and/or translation comprising: providing the cell line of any one of the foregoing embodiments; combining the cell line with a candidate agent for inhibiting coronavirus; and determining whether the candidate agent decreases the expression and/or activity of the reporter gene.

In some embodiments of the foregoing method, the cell line comprises a coronavirus reporter replicon in an in vitro transcribed RNA. In some embodiments, the cell line comprises a coronavirus replicon expressed from a vector of any one of the foregoing embodiments. In some embodiments, the candidate agent inhibits coronavirus RNA replication.

In some embodiments of the foregoing methods, the assay does not require a BSL3 facility. In some embodiments, the assay may be conducted in a BSL2 facility.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate at least one embodiment and together with the description, explain the principles described herein. The summary of the technology described above is non-limiting and other features and advantages of the technology will be apparent from the following detailed description, and from the claims.

### BRIEF DESCRIPTION OF THE DRAWINGS

FIGS. 1A-C show the construction and transcriptional analysis of a SARS-CoV-2 replicon. FIG. 1A shows a schematic diagram of reporter replicon genome structure, replication, and subgenomic (sg) mRNA production. The spike (S) and envelope/membrane (E/M) open reading frames (ORFs) in the wild type viral genome were deleted and replaced with a Luciferase-GFP fusion reporter (Luc-GFP) and Neo genes, respectively. The full-length replicon cDNA was flanked by a T7 promoter and a polyA/hepatitis D virus (HDV) ribozyme (RbZ) and T7 terminator cassette (polyA/RbZ/T7 terminator). The in vitro transcribed replicon RNA is copied by replicase complex (encoded by ORF1ab) to produce genomic or subgenomic sized negative stranded RNAs (dashed lines). The negative stranded sg RNAs are used as templates to produce the subgenomic (sg) mRNAs for expression of reporter and structural proteins. The sg mRNAs consist of the leader at 5′ (untranslated region) UTR of the genome and mRNA body sequences joined by a short, conserved sequence motif, the transcription-regulating sequence (TRS). The distribution of the TRS leader (TRS-L) and TRS body (TRS-B) are also indicated. FIG. 1B shows detection of the sg mRNA expressing Luc-GFP reporter protein. Total RNA was collected and purified at indicated times from human embryonic kidney (HEK) 293T cells (293T) electroporated with T7 polymerase transcribed replicon RNA. The mRNAs expressing Luc-GFP or β-actin were examined by RT-PCR. FIG. 1C shows GFP reporter signals produced by the wild type and RNA-dependent RNA polymerase (RdRp) mutant replicons. The A549, Calu-1, and Huh-7.5 cells were electroporated with T7 polymerase transcribed wild type replicon RNA or the RdRp D760N/D761N double mutant (negative control) replicon RNA and transferred to 384 well plates. The number of GFP positive cells in each well were counted at 30 h post electroporation.

FIGS. 2A-F show dose response curves of SARS-CoV-2 replicon reporter activity to GS-441524 and GC376. The 293T cells were electroporated with T7 polymerase transcribed replicon RNA and incubated with GS-441524 or GC376 at indicated concentrations. Percent GFP or luciferase activity reported in FIGS. 2A-D were determined by comparing GS-441524 or GC376-treated cells to DMSO-treated cells. FIGS. 2E-F show cytotoxicity of GS-441524 and GC376 to the cells.

FIG. 3 shows a schematic diagram of ORF1b and luc-GFP sequence in different Middle East respiratory syndrome (MERS) replicon constructs.

### DETAILED DESCRIPTION

### Description of Embodiments

### I. Coronavirus Replicon

Noninfectious coronavirus replicons provide increased safety and flexibility for investigators working to test candidate agents against coronaviruses, such as SARS-CoV-2, SARS-Cov-1, and Middle East Respiratory Syndrome (MERS). Replicons are self-replicating subgenomic systems in which genes encoding viral structural proteins are replaced by a reporter gene. By maintaining some genes of coronavirus, adding a reporter gene, and by creating a noninfectious variant due to deletion of at least one structural protein, investigators can use this replicon without needing BSL3 laboratories. This makes the ability to investigate candidate agents against coronavirus more accessible to a wider variety of investigators.

A coronavirus reporter replicon, such as a SARS-CoV-2, SARS-Cov-1, or MERS replicon comprises (a) one or more coronavirus (e.g. SARS-CoV-2, SARS-Cov-1, or MERS) genes encoding one or more coronavirus proteins for coronavirus viral RNA replication; and (b) at least one reporter gene. A coronavirus protein for viral RNA replication is a protein that if deleted, mutated, or inhibited, has the effect of reducing the viral RNA replication. The reporter replicon is replication competent, but is noninfectious. In some embodiments, the reporter replicon maintains all genes and genetic elements necessary for replicating full-length RNA and subgenomic RNAs.

The replicon may be a DNA molecule or an RNA molecule. While certain sequences are provided in this application in DNA or RNA form, a person of ordinary skill in the art can easily convert DNA sequences to RNA sequences and vice versa. Thus, the scope of the application and the scope of the claims embodies both DNA and RNA versions of the replicon, vectors, and other elements. In some embodiments, the replicon is a cDNA replicon made to facilitate mutagenesis and made by reverse transcribing single-stranded positive RNA from coronavirus (e.g. SARS-CoV-2, SARS-Cov-1, or MERS).

A. At Least One Coronavirus Gene Encoding a Coronavirus Protein

In some embodiments, the at least one coronavirus gene in the coronavirus reporter replicon (e.g. SARS-CoV-2, SARS-Cov-1, or MERS) encodes a target for a candidate antiviral agent. The replicon may comprise a single target or it may comprise multiple targets (i.e., it may include one or any combination of coronavirus proteins). In some embodiments, the target is at least one viral protein for coronavirus viral RNA replication (e.g. SARS-CoV-2, SARS-Cov-1, or MERS).

The SARS-CoV-2 viral RNA genome comprises open reading frame (ORF) 1a and ORF1ab, spike surface protein (S) ORF, ORF3a, envelope protein (E) ORF, membrane protein (M) ORF, ORF6, ORF7a, ORF7b, ORF8, nucleocapsid protein (N) ORF, ORF10, and 3′ UTR (Khailany, Rozhgar, A. et al. “Genomic characterization of a novel SARS-CoV-2.” Gene reports vol. 19 (2020): 100682. doi:10.1016/j.genrep.2020.100682, incorporated by reference herein). ORF1ab encodes for 16 non-structural proteins (nsp) of the replicase complex, i.e., nsp 1, 2, 3, 4, 5, 6, 7, 8, 9, 10, 11, 12, 13, 14, 15, and 16. ORF1a encodes nsp1-11 (Krichel et al. Biochem J. 2020 Mar. 13; 477(5): 1009-1019, incorporated by reference herein). S, E, M, and N ORFs encode for the structural proteins S, E, M, and N. ORFs 6, 7a, 7b, 8, and 10 encode for the accessory proteins (Romano, Maria et al. “A Structural View of SARS-CoV-2 RNA Replication Machinery: RNA Synthesis, Proofreading and Final Capping.” Cells vol. 9, 5 1267. 20 May. 2020, doi:10.3390/cells9051267; incorporated by reference herein).

The SARS-CoV-1 viral RNA genome comprises ORF1a and ORF1b, spike surface protein ORF, ORF3a, ORF3b, envelope protein (E) ORF, membrane protein (M) ORF, ORF6, ORF7a, ORF7b, ORF8a, ORF8b, nucleocapsid protein (N) ORF, OR9b and 3′ UTR (DeWit et al., “SARS and MERS: recent insights into emerging coronaviruses”, Nat Rev Microbiol. 2016 August; 14(8):523-34. doi: 10.1038/nrmicro.2016.81, incorporated by reference herein). ORF1a and ORF1b encode for 16 non-structural proteins (nsp) of the replicase complex, i.e., nsp 1, 2, 3, 4, 5, 6, 7, 8, 9, 10, 11, 12, 13, 14, 15, and 16. SARS-CoV-1 transcribes 12 subgenomic RNAs which encode the four structural proteins spike (S), envelope (E), membrane (M) and nucleocapsid (N), as well as several accessory proteins that are not involved in viral replication but either interfere with the host innate immune response or have unknown function.

The MERS viral RNA genome comprises ORF1a and ORF1b, spike surface protein ORF, ORF3, ORF4a, ORF4b, ORF5, envelope protein (E) ORF, membrane protein (M) ORF, nucleocapsid protein (N) ORF, and OR8b. (DeWit et al., “SARS and MERS: recent insights into emerging coronaviruses”, Nat Rev Microbiol. 2016 August; 14(8):523-34. doi: 10.1038/nrmicro.2016.81). ORF1a and ORF1b encode for 16 non-structural proteins (nsp) of the replicase complex, i.e., nsp 1, 2, 3, 4, 5, 6, 7, 8, 9, 10, 11, 12, 13, 14, 15, and 16. MERS transcribes 9 subgenomic RNAs which encode the four structural proteins spike (S), envelope (E), membrane (M) and nucleocapsid (N), as well as several accessory proteins that are not involved in viral replication but either interfere with the host innate immune response or have unknown function.

In some embodiments, the target for a candidate antiviral agent is an RNA-dependent RNA polymerase (RdRp). In some embodiments, RdRp is nsp12. In some embodiments, RdRp is SEQ ID NO: 9 (the wild-type RdRp). In some embodiments, RdRp may have at least one mutation compared to SEQ ID NO: 9 (the wild-type RdRp). In some embodiments, the RdRp may have a D760N mutation and/or a D761N mutation. In some embodiments, the RdRp is SEQ ID NO: 10 (the D760N RdRp mutant), 11(the D761N RdRp mutant), or 12 (the D760N/D761N RdRp mutant). In some embodiments RdRP is SEQ ID NO: 34 (RdRp from MERS). In some embodiments, RdRp is SEQ ID NO: 35 (RdRp from SARS-CoV-1).

In some embodiments, the target for a candidate antiviral agent is a protease. In some embodiments, the protease is nsp5. In some embodiments, the protease is M^{por} or 3CL^{pro}.

In some embodiments, the coronavirus reporter replicon further comprises a nucleocapsid ORF.

In some embodiments, the coronavirus reporter replicon comprises a replicase complex ORF1ab, a reporter gene, a selection gene, and a nucleocapsid gene.

In some embodiments, the coronavirus reporter replicon comprises SEQ ID NO: 2 (the T7-scv2-luc-GFP replicon). In some embodiments, the coronavirus reporter replicon comprises SEQ ID NO: 5 (the T7-scv2-nanoluc replicon). In some embodiments, the coronavirus reporter replicon comprises SEQ ID NO: 7 (the T7GG-scv2-nanoluc replicon).

In some embodiments, the coronavirus reporter replicon comprises SEQ ID NO: 28 (the T7-mcv-replicon1). In some embodiments, the coronavirus reporter replicon comprises SEQ ID NO: 29 (the T7-mcv-replicon2). In some embodiments, the coronavirus reporter replicon comprises SEQ ID NO: 30 (the T7-mcv-replicon3).

In some embodiments, the coronavirus reporter replicon comprises SEQ ID NO: 32 (the T7-scv1-luc-GFP replicon). In some embodiments, the coronavirus reporter replicon comprises SEQ ID NO: 33 (the T7-scv1-luc-GFP replicon).

In some embodiments, the coronavirus reporter replicon may have sequences for TRS-L and/or TRS-B. In some embodiments, the coronavirus reporter replicon further comprises ORF3a, ORF6, ORF7a, ORF7b, ORF8, and/or inter ORF10.

In some embodiments, the coronavirus reporter replicon does not comprise at least one functional coronavirus ORF or functional ORF from any virus. The coronavirus ORF or viral ORF missing from the coronavirus reporter replicon may be a spike ORF, an envelope ORF, or a membrane ORF, whether from SARS-CoV-2, SARS-CoV-1, MERS, or from any other virus. In some embodiments, the coronavirus reporter replicon does not comprise at least one of a functional coronavirus spike ORF, envelope ORF, or membrane ORF. In some embodiments, the coronavirus reporter replicon does not comprise a functional coronavirus spike ORF. In some embodiments, the coronavirus reporter replicon does not comprise a functional coronavirus envelope ORF. In some embodiments, the coronavirus reporter replicon does not comprise a functional coronavirus membrane ORF. In some embodiments, the coronavirus reporter replicon does not comprise at least one of functional spike ORF, envelope ORF, or membrane ORF. In some embodiments, the coronavirus reporter replicon does not comprise a functional spike ORF. In some embodiments, the coronavirus reporter replicon does not comprise a functional envelope ORF. In some embodiments, the coronavirus reporter replicon does not comprise a functional membrane ORF. In some embodiments, the coronavirus reporter replicon does not comprise a functional spike ORF, envelope ORF, or membrane ORF.

B. At Least One Reporter Gene

Adding a reporter gene to the replicon facilitates the ease of evaluating whether a candidate agent inhibits the coronavirus replicon and whether the candidate agent should be considered for further preclinical and clinical trials for treatment of coronavirus infection (e.g. SARS-CoV-2, SARS-CoV-1, or MERS).

In some embodiments, at least one reporter gene is a bioluminescent reporter gene, a fluorescent protein gene, and/or an enzymatic reporter gene. In some embodiments, the at least one reporter gene is a gene for firefly luciferase (Luc), Renilla luciferase (RLuc), NanoLuc® Luciferase Nluc, NanoLuc® Luciferase NlucP, NanoLuc® Luciferase secNluc, and/or Gaussia luciferase (GLuc). In some embodiments, the at least one reporter gene comprises firefly luciferase. In some embodiments, the at least one reporter gene is a gene for green fluorescent protein (GFP), enhanced GFP (eGFP), red fluorescent protein (RFP), mCherry, and/or Discosoma Red (DsRed). In some embodiments, the at least one reporter gene comprises GFP. In some embodiments, the at least one reporter gene is a gene for β-lactamase, β-galactosidase, and/or secreted alkaline phosphatase (SAP). In some embodiments, the at least one reporter gene comprises a fusion gene of two or more reporter genes. In some embodiments, the at least one reporter gene is a fusion gene of firefly luciferase and GFP.

C. A Selection Gene

In some embodiments, the coronavirus reporter replicon comprises a selection gene. A selection gene may comprise an antibiotic resistance gene. In some embodiments, the selection gene imparts resistance to neomycin, puromycin, blasticidin, Geneticin® (G418 sulfate), Zeocin™, and/or hygromycin B. The selection gene may be neomycin phosphotransferase gene, puromycin N-acetyltransferase gene, blasticidin-S resistance gene (bsr), blasticidin-S deaminase (bsd), blasticidin-S acetyltransferase gene (bls), bleomycin resistance gene (Sh ble), and/or hygromycin B phosphotransferase gene (hph).

### II. Vector Comprising the Coronavirus Reporter Replicon

A vector may comprise a replication competent, noninfectious coronavirus reporter replicon of the present disclosure, as described in Section I above. As discussed above, the reporter and replicon may be in DNA or RNA format. In some embodiments, the vector may include at least one promoter and/or a terminator cassette. In some embodiments, the vector is a circular nucleic acid molecule, such as a plasmid.

A variety of expression vectors can be used to express a nucleic acid molecule. Such vectors include chromosomal, episomal, and virus-derived vectors, e.g., vectors derived from bacterial plasmids, from bacteriophage, from yeast episomes, from yeast chromosomal elements, including yeast artificial chromosomes (YAC), from viruses such as baculoviruses, papovaviruses such as SV40, vaccinia viruses, adenoviruses, poxviruses, pseudorabies viruses, herpes viruses, and retroviruses. Vectors may also be derived from combinations of these sources, such as those derived from plasmid and bacteriophage genetic elements, e.g., cosmids and phagemids. In some embodiments, the vector is a bacterial artificial chromosome (BAC).

A. Promoter

In some embodiments, the vector comprising the coronavirus reporter replicon contains at least one promoter. A promoter can be situated at the 5′ end of an ORF. In some embodiments, the promoter is a T7 promoter, a T7GG promoter, a T7GGG promoter, an EF-1a promoter, a human pol I promoter, a CMV promoter, SP6, T3 promoter, and/or a CMV/T7 dual promoter. In some embodiments, the promoter is a T7 promoter. In some embodiments, the promoter is a T7GG promoter. In some embodiments, the promoter is a CMV promoter. In some embodiments, the promoter is a CMV/T7 dual promoter.

B. Terminator Cassette

In some embodiments, the vector comprising the coronavirus reporter replicon contains a terminator cassette. A terminator cassette can be situated at the 3′ end of an ORF. In some embodiments, the terminator cassette is a T7 terminator cassette.

In some embodiments, the vector comprising the coronavirus reporter replicon may have a polyA sequence and/or a virus ribozyme (RbZ) sequence, in addition to a terminator cassette. In some embodiments, the polyA sequence and/or RbZ sequence are a part of the T7 terminator (T7T) cassette, i.e., polyA/RbZ/T7 terminator. In some embodiments, the virus RbZ sequence is a hepatitis virus RbZ sequence. In some embodiments, the hepatitis virus is a hepatitis B virus (HBV) or a hepatitis D virus (HDV). In some embodiments, the vector comprising the coronavirus reporter replicon contains a polyA sequence and an HDV RbZ sequence.

C. Vector Sequences Comprising the SARS-CoV-2 Reporter Replicon

In some embodiments, the vector comprises a non-mammalian promoter and a coronavirus reporter replicon. In some embodiments, the vector is a BAC vector. In some embodiments, the vector comprises a T7 promoter and a coronavirus reporter replicon. In some embodiments, the vector comprises SEQ ID NO: 2 (the T7-scv2-luc-GFP replicon) or a sequence that is 90%, 91%, 92%, 93%, 94%, 95%, 96%, 97%, 98%, or 99% homologous to SEQ ID NO: 2 (the T7-scv2-luc-GFP replicon). In some embodiments, the vector comprises SEQ ID NO: 28 (the T7-mcv-replicon1) or a sequence that is 90%, 91%, 92%, 93%, 94%, 95%, 96%, 97%, 98%, or 99% homologous to SEQ ID NO: 28. In some embodiments, the vector comprises SEQ ID NO: 29 (the T7-mcv-replicon2) or a sequence that is 90%, 91%, 92%, 93%, 94%, 95%, 96%, 97%, 98%, or 99% homologous to SEQ ID NO: 29. In some embodiments, the vector comprises SEQ ID NO: 30 (the T7-mcv-replicon3) or a sequence that is 90%, 91%, 92%, 93%, 94%, 95%, 96%, 97%, 98%, or 99% homologous to SEQ ID NO: 30. In some embodiments, the vector comprises SEQ ID NO: 32 (the T7-scv1-luc-GFP replicon) or a sequence that is 90%, 91%, 92%, 93%, 94%, 95%, 96%, 97%, 98%, or 99% homologous to SEQ ID NO: 32. In some embodiments, the vector comprises SEQ ID NO: 33 (the T7-scv1-luc-GFP replicon) or a sequence that is 90%, 91%, 92%, 93%, 94%, 95%, 96%, 97%, 98%, or 99% homologous to SEQ ID NO: 33.

In some embodiments, the vector comprises a T7 promoter, a SARS-CoV-2 reporter replicon, a polyA/RbZ/T7 terminator and a pSMART BAC vector backbone. In some embodiments, the pSMART BAC vector backbone comprises a second promoter. In some of these embodiments, the vector then comprises a dual promoter. In some embodiments, the vector comprises a CMV/T7 dual promoter.

In some embodiments, the vector is a T7-scv2-luc-GFP replicon (i.e., SEQ ID NO: 2) in a pSMART BAC vector with a CMV promoter. In some embodiments, the vector comprises SEQ ID NO: 3 (the pSMART BAC vector with the CMV-T7-scv2-luc-GFP replicon) or a sequence that is 90%, 91%, 92%, 93%, 94%, 95%, 96%, 97%, 98%, or 99% homologous to SEQ ID NO: 3 (the pSMART BAC vector with the CMV-T7-scv2-luc-GFP replicon). In some embodiments, the vector comprises SEQ ID NO: 28 (the T7-mcv-replicon1) with a CMV promoter. In some embodiments, the vector comprises SEQ ID NO: 29 (the T7-mcv-replicon2) with a CMV promoter. In some embodiments, the coronavirus reporter replicon comprises SEQ ID NO: 30 (the T7-mcv-replicon3) with a CMV promoter. In some embodiments, the vector comprises SEQ ID NO: 32 (the T7-scv1-luc-GFP replicon) with a CMV promoter. In some embodiments, the vector comprises SEQ ID NO: 33 (the T7-scv1-luc-GFP replicon) with a CMV promoter.

In some embodiments, the vector is a T7-scv2-luc-GFP replicon (i.e., SEQ ID NO: 2) in a pSMART BAC vector without a CMV promoter. In some embodiments, the vector comprises SEQ ID NO: 13 (the pSMART BAC vector with the T7-scv2-luc-GFP replicon) or a sequence that is 90%, 91%, 92%, 93%, 94%, 95%, 96%, 97%, 98%, or 99% homologous to SEQ ID NO: 13 (the pSMART BAC vector with the T7-scv2-luc-GFP replicon). In some embodiments, the vector comprises SEQ ID NO: 28 (the T7-mcv-replicon1) in a pSMART BAC vector without a CMV promoter. In some embodiments, the vector comprises SEQ ID NO: 29 (the T7-mcv-replicon2) in a pSMART BAC vector without a CMV promoter. In some embodiments, the coronavirus reporter replicon comprises SEQ ID NO: 30 (the T7-mcv-replicon3) in a pSMART BAC vector without a CMV promoter. In some embodiments, the vector comprises SEQ ID NO: 32 (the T7-scv1-luc-GFP replicon) in a pSMART BAC vector without a CMV promoter. In some embodiments, the vector comprises SEQ ID NO: 33 (the T7-scv1-luc-GFP replicon) in a pSMART BAC vector without a CMV promoter.

In some embodiments, the vector comprises a T7GG promoter and a coronavirus reporter replicon. In some embodiments, the vector comprises SEQ ID NO: 4 (the pSMART BAC vector with the T7GG-scv2-GFP-luc replicon) or a sequence that is 90%, 91%, 92%, 93%, 94%, 95%, 96%, 97%, 98%, or 99% homologous to SEQ ID NO: 4 (the pSMART BAC vector with the T7GG-scv2-GFP-luc replicon). In some embodiments, the vector comprises SEQ ID NO: 28 (the T7-mcv-replicon1) in a pSMART BAC vector without a CMV promoter. In some embodiments, the vector comprises SEQ ID NO: 29 (the T7-mcv-replicon2) in a pSMART BAC vector without a CMV promoter. In some embodiments, the coronavirus reporter replicon comprises SEQ ID NO: 30 (the T7-mcv-replicon3) in a pSMART BAC vector without a CMV promoter. In some embodiments, the vector comprises SEQ ID NO: 32 (the T7-scv1-luc-GFP replicon) in a pSMART BAC vector without a CMV promoter. In some embodiments, the vector comprises SEQ ID NO: 33 (the T7-scv1-luc-GFP replicon) in a pSMART BAC vector without a CMV promoter.

In some embodiments, the vector comprises a coronavirus reporter replicon with nanoluciferase reporter gene. In some embodiments, the vector comprises SEQ ID NO: 6 (the pSMART BAC with the CMV-T7-scv2-nanoluc replicon) or a sequence that is 90%, 91%, 92%, 93%, 94%, 95%, 96%, 97%, 98%, or 99% homologous to SEQ ID NO: 6 (the pSMART BAC with the CMV-T7-scv2-nanoluc replicon).

In some embodiments, the coronavirus reporter replicon comprises SEQ ID NO: 28 (the T7-mcv-replicon1). In some embodiments, the coronavirus reporter replicon comprises SEQ ID NO: 29 (the T7-mcv-replicon2). In some embodiments, the coronavirus reporter replicon comprises SEQ ID NO: 30 (the T7-mcv-replicon3).

In some embodiments, the coronavirus reporter replicon comprises SEQ ID NO: 32 (the T7-scv1-luc-GFP replicon). In some embodiments, the coronavirus reporter replicon comprises SEQ ID NO: 33 (the T7-scv1-luc-GFP replicon).

In some embodiments, the vector does not comprise a gene for the RdRp double mutant D760N/D761N.

D. RNA and DNA Vectors

In some embodiments, the vector is a DNA vector. In some embodiments, the vector is an RNA vector. In situations where the replicon is an DNA replicon, it may be transcribed and inserted into an RNA vector. More specifically, in some embodiments, the coronavirus reporter replicon RNA is prepared using in vitro transcription. In some embodiments, the coronavirus reporter replicon RNA is transcribed from a linearized vector. In some embodiments, the RNA is transcribed from a SARS-CoV-2 reporter DNA replicon of SEQ ID NO: 2 (the T7-scv2-luc-GFP replicon), 4 (the T7GG-scv2-luc-GFP replicon), 5 (the T7-scv2-nanoluc replicon), or 7 (the T7GG-scv2-nanoluc replicon). In some embodiments, the RNA is transcribed from a linearized vector of SEQ ID NO: 3 (the pSMART BAC vector with the CMV-T7-scv2-luc-GFP replicon). In some embodiments, the RNA is transcribed from a linearized vector of SEQ ID NO: 4 (the pSMART BAC vector with the T7GG-scv2-GFP-luc replicon). In some embodiments, the RNA is transcribed from a linearized vector of SEQ ID NO: 6 (the pSMART BAC vector with the CMV-T7-scv2-nanoluc replicon). In some embodiments, the RNA is transcribed from a linearized vector of SEQ ID NO: 13 (the pSMART BAC vector with the T7-scv2-luc-GFP replicon). In some embodiments, the RNA is transcribed from a linearized vector of SEQ ID NO: 28 (the T7-mcv-replicon1). In some embodiments, the RNA is transcribed from a linearized vector of SEQ ID NO: 29 (the T7-mcv-replicon2). In some embodiments, the RNA is transcribed from a linearized vector of SEQ ID NO: 30 (the T7-mcv-replicon3). In some embodiments, the RNA is transcribed from a linearized vector of SEQ ID NO: 32 (the T7-scv1-luc-GFP replicon). In some embodiments, the RNA is transcribed from a linearized vector of SEQ ID NO: 33 (the T7-scv1-luc-GFP replicon).

### III. Cell Lines

Many cell lines (i.e., host cell lines) can be used to express the coronavirus reporter replicon. Many cell lines and related methods may be used to support challenges in developing antivirals capable of protecting a broad range of host cells, such as different transfection efficiencies and candidate antiviral potencies. In some embodiments, the cell line is a mammalian cell line. In some embodiments, the cell line is human embryonic kidney (HEK) 293T (293T), Huh7.5, Calu-1, A549, or Vero. In some embodiments, the cell line is not Vero.

In some embodiments, host cells can include bacterial cells including, but not limited to, E. coli, Streptomyces, and Salmonella typhimurium, eukaryotic cells including, but not limited to, yeast, insect cells, such as Drosophila, animal cells, such as Huh-7, HeLa, COS, HEK 293, HEK 293T, MT-2T, CEM-SS, and CHO cells, and plant cells.

Many methods may be used to transfect cells with a coronavirus reporter replicon. In some embodiments, transfection is achieved by electroporation, lipotransfection, microinjection, gene gun, impalefection, hydrostatic pressure, continuous infusion, sonication, polymeric gene carriers, or other methods.

After transfection of cells with a coronavirus reporter replicon, replicon mRNA production in cells may be confirmed by RT-PCR. Primers may be generated using standard primer generation tools. In some embodiments, the reporter gene (such as Luc-GFP sg mRNA) may be detected by RT-PCR using a forward primer in the leader sequence of the 5′ UTR and a reverse primer in the reporter (for example, Luc-GFP) coding region. In some embodiments, the reporter gene (such as Luc-GFP sg mRNA) is detected after at least 12 h post transfection. In some embodiments, the presence of the reporter gene (such as Luc-GFP sg) mRNA is indicated by the detection of the reporter signal (in one embodiment, GFP fluorescence) in the cell. In some embodiments, the presence of the reporter gene (for example, Luc-GFP sg mRNA) is indicated by the detection of reporter activity (for example, luciferase activity). Other primers, other reporter signals, and other reporter genes may be used as described herein.

In some embodiments, a cell line is transfected with a vector comprising a coronavirus reporter DNA replicon. In some embodiments, the vector comprises SEQ ID NO: 2 (the T7-scv2-luc-GFP replicon), SEQ ID NO: 3 (the pSMART BAC vector with the CMV-T7-scv2-luc-GFP replicon), SEQ ID NO: 4 (the pSMART BAC vector with the T7GG-scv2-luc-GFP replicon), SEQ ID NO: 5 (the T7-scv2-nanoluc replicon), SEQ ID NO: 6 (the pSMART BAC vector with the CMV-T7-scv2-nanoluc replicon), SEQ ID NO: 7 (the T7GG-scv2-nanoluc replicon), or SEQ ID NO: 13 (the pSMART BAC vector with the T7-scv2-luc-GFP replicon), or a sequence that is 90%, 91%, 92%, 93%, 94%, 95%, 96%, 97%, 98%, or 99% homologous to any of those sequences. In some embodiments, a cell line is transfected with a vector of SEQ ID NO: 3 (the pSMART BAC vector with the CMV-T7-scv2-luc-GFP replicon). In some embodiments, a cell line is transfected with a vector of SEQ ID NO: 4 (the pSMART BAC with the T7GG-scv2-luc-GFP replicon). In some embodiments, a cell line is transfected with a vector of SEQ ID NO: 6 (the pSMART BAC vector with the CMV-T7-scv2-nanoluc replicon). In some embodiments, a cell line is transfected with a vector of SEQ ID NO: 13 (the pSMART BAC vector with the T7-scv2-luc-GFP replicon). In some embodiments, the vector comprises SEQ ID NO: 28 (the T7-mcv-replicon1) or a sequence that is 90%, 91%, 92%, 93%, 94%, 95%, 96%, 97%, 98%, or 99% homologous to SEQ ID NO: 28. In some embodiments, the vector comprises SEQ ID NO: 29 (the T7-mcv-replicon2) or a sequence that is 90%, 91%, 92%, 93%, 94%, 95%, 96%, 97%, 98%, or 99% homologous to SEQ ID NO: 29. In some embodiments, the vector comprises SEQ ID NO: 30 (the T7-mcv-replicon3) or a sequence that is 90%, 91%, 92%, 93%, 94%, 95%, 96%, 97%, 98%, or 99% homologous to SEQ ID NO: 30. In some embodiments, the vector comprises SEQ ID NO: 32 (the T7-scv1-luc-GFP replicon) or a sequence that is 90%, 91%, 92%, 93%, 94%, 95%, 96%, 97%, 98%, or 99% homologous to SEQ ID NO: 32. In some embodiments, the vector comprises SEQ ID NO: 33 (the T7-scv1-luc-GFP replicon) or a sequence that is 90%, 91%, 92%, 93%, 94%, 95%, 96%, 97%, 98%, or 99% homologous to SEQ ID NO: 33.

In some embodiments, a cell line is transfected with an in vitro transcribed RNA of the SARS-CoV-2 reporter replicon. In some embodiments, the in vitro transcribed RNA is the full-length replicon RNA. In some embodiments, the in vitro transcribed RNA comprises an RNA equivalent of a DNA sequence comprising SEQ ID NO: 2 (the T7-scv2-luc-GFP replicon), SEQ ID NO: 3 (the pSMART BAC vector with the CMV-T7-scv2-luc-GFP replicon), SEQ ID NO: 4 (the pSMART BAC with the T7GG-scv2-luc-GFP replicon), SEQ ID NO: 5 (the T7-scv2-nanoluc replicon), SEQ ID NO: 6 (the pSMART BAC vector with the CMV-T7-scv2-nanoluc replicon), SEQ ID NO: 7 (the T7GG-scv2-nanoluc replicon), SEQ ID NO: 13 (the pSMART BAC vector with the T7-scv2-luc-GFP replicon), SEQ ID NO: 28 (the T7-mcv-replicon1), SEQ ID NO: 29 (the T7-mcv-replicon2), SEQ ID NO: 30 (the T7-mcv-replicon3), SEQ ID NO: 32 (the T7-scv1-luc-GFP replicon), or SEQ ID NO: 33 (the T7-scv1-luc-GFP replicon) or a sequence that is 90%, 91%, 92%, 93%, 94%, 95%, 96%, 97%, 98%, or 99% homologous to any of those sequences.

In some embodiments, a cell line is transfected with an RNA replicon with the RNA equivalent of a DNA sequence comprising SEQ ID NO: 2 (the T7-scv2-luc-GFP replicon), SEQ ID NO: 3 (the pSMART BAC vector with the CMV-T7-scv2-luc-GFP replicon), SEQ ID NO: 4 (the pSMART BAC vector with the T7GG-scv2-luc-GFP replicon), SEQ ID NO: 5 (the T7-scv2-nanoluc replicon), SEQ ID NO: 6 (the pSMART BAC vector with the CMV-T7-scv2-nanoluc replicon), SEQ ID NO: 7 (the T7GG-scv2-nanoluc replicon), SEQ ID NO: 13 (the pSMART BAC vector with the T7-scv2-luc-GFP replicon), SEQ ID NO: 28 (the T7-mcv-replicon1), SEQ ID NO: 29 (the T7-mcv-replicon2), SEQ ID NO: 30 (the T7-mcv-replicon3), SEQ ID NO: 32 (the T7-scv1-luc-GFP replicon), or SEQ ID NO: 33 (the T7-scv1-luc-GFP replicon) or a sequence that is 90%, 91%, 92%, 93%, 94%, 95%, 96%, 97%, 98%, or 99% homologous to any of those sequences.

### IV. Methods of Assaying Candidate Agents

In many embodiments, a method of assaying a candidate agent for inhibition of coronavirus replication and/or translation comprises (a) providing the cell line transfected with a coronavirus reporter replicon; (b) combining the cell line with a candidate agent for inhibiting coronavirus; and (c) determining whether the candidate agent decreases the expression and/or activity of the reporter gene. A wide variety of candidate agents may be tested that are potentially capable of inhibiting one or more of the genes or proteins present in the coronavirus reporter replicon.

Inhibitors of viral replication and/or translation may decrease the expression and activity of the replicon reporter protein. In many embodiments, a high throughput screen is used to identify candidate agents as inhibitors against coronavirus or any virus, wherein the screen is carried out using microplates and robotic liquid dispensers. Candidate agents and cells that were transfected with reporter replicon are incubated for a certain period of time before the candidate agents are evaluated for antiviral activity, depending on the agent selected and the growth rate of the cells chosen for the assay. In many embodiments, candidate agents are evaluated for antiviral activity after at least 24 h of incubation. In some embodiments, the candidate agents are evaluated after at least 30 h of incubation.

In many embodiments, the microplates are 96-well plates, 384-well plates, or 1536-well plates. In many embodiments, the microplates are poly-D lysine-coated plates or cell culture-treated, flat-bottom microplates.

In some embodiments, candidate agents and controls are added to the microplate first, and the cells are added second. In some embodiments, the cells are added to the microplate first, and the candidate agents and controls are added second. The agents and the cells may also be added simultaneously.

In many embodiments, each candidate agent is tested in serial dilution, such as a 10-point serial 3-fold dilution. In some embodiments, a suitable vehicle may be used as a negative control, wherein the vehicle is a solution that solubilizes the candidate agent. In some embodiments, DMSO may be used as a negative control. In some embodiments, a known antiviral compound that does not inhibit viral RNA synthesis and/or protein translation may be used as a negative control. In some embodiments, the known antiviral compound may inhibit host cell entry or endosome entry. In some of these embodiments, nafamostat (nafamostat mesylate; nafamostate mesilate; Fusan), camostat (camostat mesylate; camostate mesylate; Foipan), apilimod (STA-5326), and/or relacatib (462795; GSK-462795; SB-462795) may be used as a negative control. In some embodiments, a known antiviral compound may be used as a positive control. In some embodiments, remdesivir may be used as a positive control. In some embodiments, GS-441524 may be used as a positive control.

In many embodiments, the cells are transfected with a reporter replicon before being added to the wells. In many embodiments, the cells may be transfected with a SARS-CoV-2 reporter replicon of the present disclosure or a reporter replicon of any virus. Reporter replicon transfection may be carried out according to established tools and methods known to a practitioner of ordinary skill, such as the MaxCyte Flow Electroporation® technology. In many embodiments, the cells are added to the microplate after transfection with a reporter replicon.

Depending on a variety of parameters, such as the type of microplate and the type of cells being used, a predetermined number of cells is added into each well in a plate. In some embodiments, 20,000 transfected cells are added to each well. Depending on the cell line being used, specific culture conditions may be used to incubate cells with the candidate agents. In some embodiments, the incubation conditions are 37° C., 5% CO₂, and 90% relative humidity.

Antiviral activity is determined by reduction in the number of GFP positive cells or luciferase activity (or other reporter signal) in the cells treated with candidate agent when compared to those of vehicle-treated (negative control) cells. In many embodiments, antiviral activity comprises inhibition of SARS-CoV-2 replication and/or translation. A variety of methods can be used to assess the candidate agents for antiviral activity. In some embodiments, the cells are analyzed for GFP fluorescence (or other reporter signal). In some of these embodiments, a microplate cytometer such as an Acumen® eX3 fluorescence microplate cytometer may be used for GFP analysis. In other embodiments, the cells are analyzed for luciferase activity. In some of these embodiments, plate reader such as an EnVision® multimode plate reader may be used for luciferase analysis.

In many embodiments, EC50 is determined for candidate agents, wherein the EC50 is the concentration of the candidate agent that provides response that is halfway between the baseline and the maximum response. In many embodiments, the baseline is provided by cells treated with a vehicle, such as DMSO. In many embodiments, the maximum response is provided by cells treated with a positive control, such as remdesivir or GS-441524. In many embodiments, EC50 was determined by using a non-linear 4-parameter curve fitting model in ActivityBase.

Candidate agents are also evaluated using cytotoxicity assays which measure loss of some cellular or intercellular structure and/or function, including lethal cytotoxicity. Candidate agents may adversely affect cell health and interfere indirectly with the activity of the replicon reporter protein, producing false-positive hits in the antiviral screen. Thus, candidate agents are evaluated for cytotoxicity in parallel with inhibition of coronavirus replication and/or translation. In many embodiments, cytotoxicity methods may comprise enzyme leakage assays, membrane impermeable dyes, and/or amine-reactive dyes.

In some embodiments, the candidate agent inhibits coronavirus protein translation and/or RNA replication. In some embodiments, the candidate agent is an RNA-dependent RNA polymerase inhibitor or a main protease inhibitor or a main protease inhibitor. In some embodiments, the candidate agent is the main protease inhibitor PF-00835231, GC376, or Z-LVG-DMF.

A. Targets for Candidate Agents

A candidate agent may target a protein expressed from a coronavirus gene (e.g. a SARS-CoV-2 gene, a SARS-CoV-1 gene, or a MERS gene). Alternatively, a candidate agent may target any of a plurality of nucleic acid sequences associated with coronavirus.

In order to test candidate antiviral agents, the coronavirus reporter replicon may comprise at least one nucleic acid sequence that is a target for at least one agent. Upon cell entry, the coronavirus genomic RNA is replicated for genome synthesis or transcribed and translated to produce proteins from open reading frames (ORFs).

In some embodiments, the at least one target for at least one agent is an RNA sequence chosen from coronavirus genomic RNA, full-length mRNA, or subgenomic (sg) mRNA. In many embodiments, the genomic RNA, full-length mRNA, or subgenomic (sg) mRNA is transcribed from a coronavirus reporter replicon. In many embodiments, the transcription of genomic RNA, full-length mRNA, or subgenomic (sg) mRNA can take place in vitro or within a host cell.

In some embodiments, the at least one target for at least one agent is a DNA sequence. In some embodiments, the DNA sequence is produced by reverse transcription of a coronavirus genomic RNA, full-length mRNA, or sg mRNA. In some embodiments, the DNA sequence is a cDNA sequence. In many embodiments, the coronavirus reporter replicon comprises a cDNA sequence.

V. Noninfectious Coronavirus Replicons that Provide Biological Safety

Biological Safety Levels are a series of protections designed to protect laboratory personnel as well as the surrounding environment and community. A BSL3 laboratory typically includes work on microbes that are either indigenous or exotic, and can cause serious or potentially lethal disease through inhalation. The SARS-CoV-2, SARS-CoV-1, and MERS viruses are examples of a microbe that requires a Biological Safety Level 3 (BSL3) laboratory. The quest for rapid identification of new drugs against SARS-CoV-2 and other coronaviruses is hampered by the lack of a robust and high throughput antiviral assay that can be performed without BSL3 restrictions.

The present disclosure provides noninfectious coronavirus replicons and related methods that do not require a BSL3 laboratory. In some embodiments, the method of assaying candidate agents for inhibition of coronavirus replication and/or translation does not require a BSL3 facility. In some embodiments, the replicon may be used in a BSL2 facility.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the term “about” in quantitative terms refers to plus or minus 10% of the value it modifies (rounded up to the nearest whole number if the value is not sub-dividable, such as a number of molecules or nucleotides).

The term “candidate antiviral agent,” refers to any compound that may possess antiviral activity.

A “coding sequence” or “open reading frame” is a nucleotide sequence that is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus.

The term “fluorescent protein” refers to a protein that emits light at some wavelength after excitation by light at another wavelength. Exemplary fluorescent proteins that emit in the green spectrum range include, but are not limited to: green fluorescent protein (GFP); enhanced GFP (eGFP); superfolder GFP; AcGFP1; and ZsGreen1. Exemplary fluorescent proteins that emit light in the blue spectrum range include, but are not limited to: enhanced blue fluorescent protein (EBFP), EBFP2, Azurite, and mKalama. Exemplary fluorescent proteins that emit light in the cyan spectrum range include, but are not limited to: cyan fluorescent protein (CFP); enhanced CFP (ECFP); Cerulean; mHoneydew; and CyPet. Exemplary fluorescent proteins that emit light in the yellow spectrum range include, but are not limited to: yellow fluorescent protein (YFP); Citrine; Venus; mBanana; ZsYellow 1; and Ypet. Exemplary fluorescent proteins that emit in the orange spectrum range include, but are not limited to: mOrange; tdTomato; Exemplary fluorescent proteins that emit light in the red and far-red spectrum range include, but are not limited to: DsRed; DsRed-monomer; DsRed-Express2; mRFPi; mCherry; mStrawberry; mRaspberry; niPluni; E2-Crimson; iRFP670; iRFP682; iRFP702; iRFP720. Exemplary listings of fluorescent proteins and their characteristics may be found in Day and Davidson, Chem Soc Rev 2009 October; 38(10): 2887-2921, incorporated herein by reference.

Fluorescent proteins may include chimeric combinations of fluorescent proteins that transfer and receive energy through fluorescent resonance energy transfer (FRET) when exposed to a particular wavelength of light. In some embodiments, an acceptor in a FRET pair may emit light at a certain wavelength after accepting energy from a donor molecule exposed to another wavelength of light. Exemplary chimeric FRET pairs, include, but are not limited to ECFP-EYFP; mTurquoise2-SeYFP; EGFP-mCherry; and Clover-mRuby. In some embodiments, the acceptor molecule of chimeric fluorescent molecule may quench the light emission of a donor molecule exposed to its preferred wavelength of light. Quenching between different portions of chimeric fluorescent proteins may occur using a photoactivatable acceptor. For example, a chimeric fluorescent protein may include a photoactivatable GFP that can then quench photoemission by CFP. Examples of FRET proteins are discussed in Ehldebrandt et al., Sensors (Basel). 2016 September; 16(9): 1488, incorporated herein by reference.

The term “functional” in reference to an ORF means that the ORF is capable of encoding the referenced protein, e.g., a “functional” SARS-CoV-2 spike ORF is capable of encoding the SARS-CoV-2 spike protein.

The term “noninfectious,” refers to the inability of viral components to be transmitted from organism to organism, from cell to cell, or from cell to organism.

The term “nucleic acid” molecule generally refers to any polyribonucleotide or polydeoxribonucleotide, which may be unmodified RNA or DNA or modified RNA or DNA. “Nucleic acids” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “nucleic acid” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “nucleic acid” embraces chemically, enzymatically or metabolically modified forms of polynucleotides as typically found in nature, as well as the chemical forms of DNA and RNA characteristic of viruses and cells.

In addition, the term “DNA” refers only to the primary and secondary structure of the molecule, and does not limit it to any particular tertiary forms. Thus, the term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

The term “RNA” refers to the polymeric form of ribonucleotides in its either single-stranded form or a double-stranded helix form. In discussing the structure of particular RNA molecules, sequence may be described herein according to the normal convention of giving the sequence in the 5′ to 3′ direction.

A “promoter sequence” is a DNA regulatory region capable of binding RNA polymerase in a cell and initiating transcription of a coding sequence. For purposes of defining the present invention, a promoter sequence is bound at its 3′ terminus by the transcription initiation site and extends upstream (5′ direction) to include the minimum number of bases or elements necessary to initiate transcription at levels detectable above background. Within the promoter sequence will be found a transcription initiation site, conveniently defined by mapping with nuclease S1, as well as protein binding domains (consensus sequences) responsible for the binding of RNA polymerase. Prokaryotic promoters contain −10 and −35 consensus sequences. A promoter can also be used to refer to RNA sequences or structures in RNA virus replication.

The term “replication competent,” refers to the ability of a replicon to generate new copies of itself due to possessing genes necessary for self-generation.

A “replicon” is any genetic element (e.g., plasmid, chromosome, viral RNA) that functions as an autonomous unit of DNA or RNA replication (i.e. self-replicating). A replicon may originate from a viral genome, and may contain viral non-structural genes for viral genome replication with one or more structural proteins deleted or replaced by genes foreign to the wild type viral genome.

A “reporter gene” is a gene encoding a protein that is detectable by fluorescence, luminescence, color change, enzyme assay, or histochemistry. For example, a fluorescent reporter protein encoded by a reporter gene may be a fluorescent protein that fluoresces when exposed to a certain wavelength of light (e.g., GFP). A reporter protein may be an enzyme that catalyzes a reaction with a substrate to produce an observable change in that substrate. Enzymes such as luciferase (exemplary substrate luciferin) or β-lactamase (exemplary substrate CCF4) can cause luminescence or allow fluorescence on substrate cleavage, and enzymes such as β-galactosidase (exemplary substrate X-gal (5-bromo-4-chloro-3-indolyl-P-D-galactopyranoside)) and secreted alkaline phosphatase (exemplary substrate PNPP (p-Nitrophenyl Phosphate, Disodium Salt)) can result in a visualizable precipitate upon substrate cleavage. In some embodiments, a reporter protein is detectable by an antibody binding interaction.

A “vector” is a DNA (or RNA), such as a plasmid, phage, or cosmid, to which another DNA (or RNA) segment may be attached so as to bring about the replication, transcription, expression, or integration of the attached segment.

As used herein, the term “homology” refers to the overall relatedness between polymeric molecules, e.g. between nucleic acid molecules (e.g. DNA molecules) and/or between polypeptide molecules. Polymeric molecules (e.g. nucleic acid molecules (e.g. DNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

### EXAMPLES

The following examples are meant to be illustrative and should not be construed as further limiting. The contents of the figures and all references, patents, and published patent applications cited throughout this application are expressly incorporated herein by reference.

### Example 1: A Replication Competent, Noninfectious SARS-CoV-2 DNA Replicon in Cells

A. Methods

1. Construction of the SARS-CoV-2 Replicon

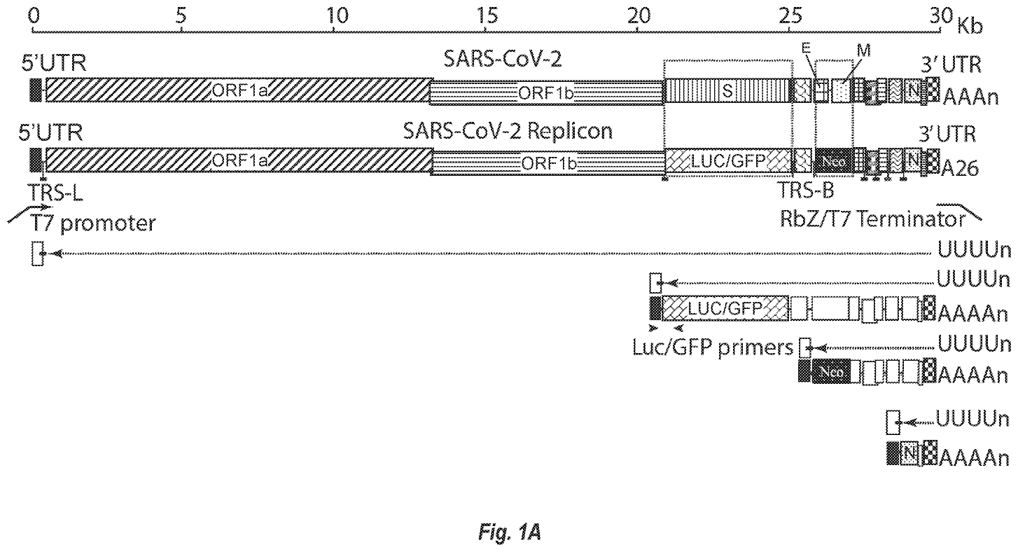

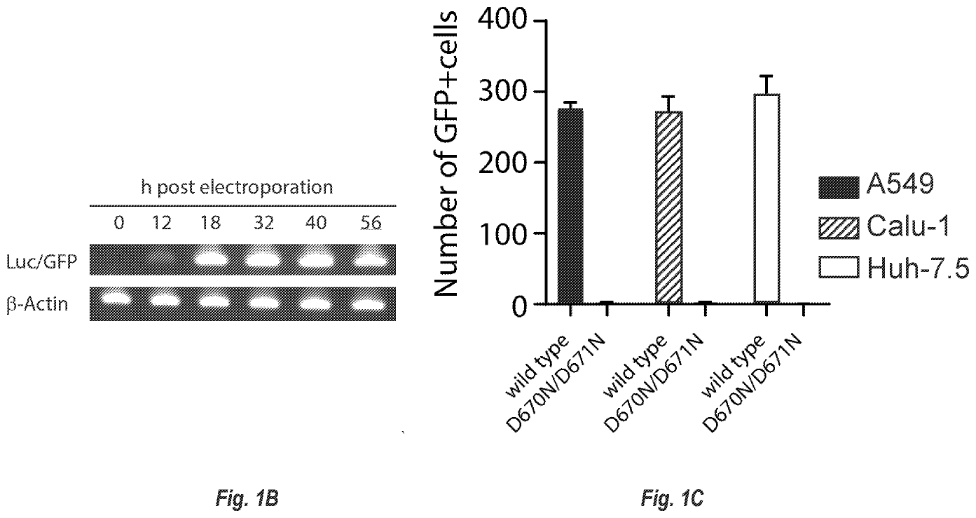

The replicon is based on severe acute respiratory syndrome coronavirus 2 isolate Wuhan-Hu-1 (GenBank: NC045512). The SARS-CoV-2 spike (S) protein open reading frame (ORF) was deleted and replaced with a GFP-firefly luciferase fusion reporter (Luc-GFP). The sequences of envelope (E), membrane (M) and their intergenic region were also deleted and replaced with a neomycin resistance gene (Neo) (FIG. 1A). The replicon maintained all the genes and genetic elements necessary for replicating the full-length and subgenomic (sg) RNAs, including replicase complex ORF1ab. Deletion of the structural genes E and M rendered the replicon defective in producing progeny virions. (The N structural gene remains in the replicon). The replicon was linked to a T7 (5′ TAATACGACTCACTATAG 3′) promoter upstream of the 5′ UTR. The T7 RNA polymerase initiates transcription at the underlined G in the promoter sequence. A cassette containing a 26-nucleotide polyA, a HDV ribozyme and T7 terminator was inserted at the 3′ end of the replicon.

The full-length SARS-CoV-2 replicon is −28 k nucleotides long. Five fragments spanning the T7 promoter, full-length SARS-CoV-2, polyA/RbZ/T7 terminator (FIG. 1A), named F1 to F5, with −30 bps overlap were synthesized by Genewiz (South Plainfield, NJ). pSMART BAC vector (Lucigen) was digested with Not I. F1 and F5 fragments were digested with Mlu I. Equimolar amounts of linearized pSMART BAC vector, F1, and F5 were ligated using Gibson Assembly kit (NEB) according to manufacturer's instruction, resulting in pSMART BAC F(1,5). Equimolar amounts of pSMART BAC F(1,5) digested with Aat II and Asc I, and F2 and F4 digested with Mlu I, were ligated using Gibson Assembly kit, resulting in pSMART BAC F(1,2,4,5). Finally, pSMART BAC F(1,2,4,5) digested with Aat II and Asc I, and F3 digested with Swa I were ligated together using Gibson Assembly Kit, resulting in the full-length noninfectious SARS-Cov-2 replicon construct SEQ ID NO: 13 (the pSMART BAC vector with T7-scv2-luc-GFP replicon).

A CMV promoter fragment was amplified from the pcDNA3.1 vector by PCR using the forward primer SEQ ID NO: 25 and the reverse primer SEQ ID NO: 26. The construct SEQ ID NO: 13 (the pSMART BAC vector with T7-scv2-luc-GFP replicon) was digested with SwaI and ligated with the CMV promoter fragment using Gibson Assembly Kit, resulting in a pSMART BAC vector with CMV-T7-scv2-luc-GFP replicon, SEQ ID NO: 3.

2. Transcription of the SARS-CoV-2 Replicon RNA

The construct SEQ ID NO: 13 (the pSMART BAC vector with the T7-scv2-luc-GFP replicon) was linearized with Swa I (NEB) digestion, then purified by phenol/chloroform extraction, precipitated with 2 volumes of ethanol, and dissolved in nuclease-free water. The mMESSAGE mMACHINE T7 ultra transcription kit (Invitrogen), which includes a cap analogue Anti-Reverse Cap Analogue (ARCA), was used to generate the replicon RNAs in the correct orientation from the linearized vector according to manufacturer's instruction. Briefly, 100 μl of T7 transcription reaction, containing 4 μg of linearized BAC and 15 μl of extra GTP, was incubated at 37° C. for 2.5 h to increase the length of the transcripts. After incubation, 5 μl of TURBO DNase was added and the reaction was incubated at 37° C. for 15 min to digest DNA. The resulting RNA was purified by Monarch RNA cleanup kit (NEB).

3. Electroporation of 293T Cells with the SARS-CoV-2 Replicon RNA

The cells were harvested using TrypLE Select (Thermofisher Scientific), washed three times with PBS and resuspended in MaxCyte electroporation buffer to 1×108 cells/ml. For 1×106 of cells, 1 μg of replicon RNA was added into resuspended cells. The mixture was immediately transferred to a MaxCyte processing assembly (PA) (OC-100, OC-400 or R-1000) and electroporation was carried out by MaxCyte STX with the pre-loaded programs for transfecting different types of cells. After resting in PA for 20 min, transfected cells were transferred into 30 ml of completed media. After counting and adjusting cell density, the RNA-electroporated cells were plated into 384-well compound assay plates.

4. Detection of Sg mRNA Expressing Luc-GFP Fusion Protein

To confirm the synthesis of the Luc-GFP sg mRNAs, we performed RT-PCR analysis on the transcripts in the electroporated cells. Total RNAs were extracted from 1.2×106 cells by RNeasy mini kit (Qiagen) at 0, 12, 18, 32, 40, and 56 h after electroporation. cDNA was synthesized and amplified a SuperScript™ IV One-Step RT-PCR kit (ThermoFisher Scientific; catalog #12594025) according to manufacturer's instructions. The PCR products were analyzed on an E-Gel 1% agarose gel (ThermoFisher Scientific).

The forward and reverse primers used to amplify the sg mRNA were: 5′-AGGTTTATACCTTCCCAGGT-3′ (SEQ ID NO: 17) and 5′-TTTGTATTCAGCCCATAGCG-3′ (SEQ ID NO: 18), and the sequences of β-actin primer set were 5′-GAGCACAGAGCCTCGCCTTT-3′ (SEQ ID NO: 19) and 5′-TGGGGTACTTCAGGGTGAGG-3′ (SEQ ID NO: 20). The primers annealed to the leader sequence of 5′ UTR and the Luc-GFP coding region, respectively (FIG. 1A). This resulted in the primer pair only amplifying Luc-GFP sgRNA.

B. Results

1. Production of the SARS-CoV-2 Replicon as Reported by GFP

Following electroporation of replicon RNA, GFP signal was detected at 12 h. According to the coronavirus replication model (Sawicki, Stanley G et al. “A contemporary view of coronavirus transcription.” Journal of virology vol. 81, 1 (2007): 20-9. doi:10.1128/JVI.01358-06), the viral genome is copied by replicase complex, both continuously to produce minus-strand templates for genome RNA synthesis and discontinuously to produce minus-strand templates for sg mRNA synthesis. These two types RNAs have also been reported recently in the SARS-CoV-2 infected cells (Kim, Dongwan et al. “The Architecture of SARS-CoV-2 Transcriptome.” Cell vol. 181, 4 (2020): 914-921.e10. doi:10.1016/j.cell.2020.04.011). The structural proteins S, E, M and nucleocapsid (N) are encoded by sg mRNAs (FIG. 1A). Therefore, GFP in the replicon RNA transfected cells is most likely also expressed by the sg mRNAs.

2. Luc-GFP Sg mRNA was Detected by RT-PCR

Consistent with the GFP expression, the Luc-GFP sg mRNA was also detected at 12 h post electroporation (FIG. 1B). This indicated that the in vitro transcribed replicon RNA is capable of self-amplifying.

### Example 2: Reporter Signal from Active Replicon RNA Replication

A. Methods

1. Construction of the RdRp Mutant Replicon

To investigate whether the GFP reporter signal was entirely generated by replicating RNAs, two point mutations, D760N and D761N, were introduced to RdRp (RNA-dependent RNA polymerase) in the SARS-CoV-2 replicon cDNA. The combination of the two mutations inactivates the RdRp and leads to complete loss of native ribonucleotide incorporation (Gordon, Calvin J et al. “Remdesivir is a direct-acting antiviral that inhibits RNA-dependent RNA polymerase from severe acute respiratory syndrome coronavirus 2 with high potency.” The Journal of Biological Chemistry vol. 295, 20 (2020): 6785-6797. doi:10.1074/jbc.RA120.013679). Thus, this construct was prepared and used as a negative control.

The RdRp mutations were made to the replicon by BAC recombineering (Murray, E et al. “Persistent replication of hepatitis C virus replicons expressing the beta-lactamase reporter in subpopulations of highly permissive Huh7 cells.” J Virol. vol. 77, 2928-2935 (2003): doi:10.1128/jvi.77.5.2928-2935.2003). An ampicillin (Amp) cassette was introduced to the RdRp D760 and D761 mutation site, followed by Gibson assembly approach to replace the Amp cassette with the D760N and D761N (D760N/D761N) mutations. The pSMART BAC vector with the CMV-T7-scv2-luc-GFP replicon (SEQ ID NO: 3) was transformed into SW102 E. coli host which expresses the bacterio-phage λ Red genes gam, bet and exo which are controlled by is repressor cI857. The Red enzymes are able to mediate homologous recombination between DNA fragments as short as 30 bp. A fragment containing an Amp cassette flanked by Asc I restriction sites and adjacent homologous sequence was amplified by PCR using primers 5′-TTTGTGAAT GAGTTTTACGCATATTTGCGTAAACATTTCTCAATGATGATACTCTCTGGCGCGC CGGAACCCCTATTTGTTTATT-3′ (SEQ ID NO: 21) and 5′-CATCTTAATGAAGTCT GTGAATTGCAAAGAACACAAGCCCCAACAGCCTGTAAGACTGGCGCGCCTTACC AATGCTTAATCAGTG-3′ (SEQ ID NO: 22) using plasmid pcDNA3.1(+) as a template. The PCR fragment was then digested with Dpn I at 37° C. for 1 h and purified using the QIAquick PCR purification kit (Qiagen). To prepare the competent SW102 containing the pSMART BAC vector with the CMV-T7-scv2-luc-GFP replicon (SEQ ID NO: 3), the E. coli host was first cultured in LB broth with 12.5 μg/ml of chloramphenicol for 4 h to reach log phase at 32° C., followed by heat shock treatment at 42° C. for 15 minutes to induce expression of λ Red genes. After 3 washes with ice-cold water, 100 ng of PCR fragment containing Amp cassette was electroporated into the competent cells. After 2 h of recovery at 32° C., the electroporated cells were plated onto LB plates containing 100 μg/ml carbenicillin and 12.5 μg/ml chloramphenicol, and then cultured overnight at 32° C. Correct clones were identified by colony PCR followed by restriction enzyme digestion. The DNA comprising pSMART BAC vector with the CMV-T7-scv2-luc-GFP replicon and Amp-resistance gene (pSMART BAC-T7-scv2-replicon-AMP DNA) was prepared using NucleoBond BAC 100 kit (Takara Cat #740579). A fragment containing the RdRp D760N/D761N mutation sequence was amplified by PCR using primers 5′-CGTAAACATTTCTCAATGATGATACTCTCTAACAATGCTGTTGTGTGTTTCAATAG-3′ (SEQ ID NO: 23) and 5′-CAAAGAACACAAGCCCCAACAGCCTGTAAGACTG TATGCGGTGTGTACATAGC-3′ (SEQ ID NO: 24). pSMART BAC-T7-scv2-replicon-AMP DNA was digested with Asc I and then ligated to the fragment containing the RdRp D760N/D761N mutations using Gibson Assembly kit resulting in pSMARTBAC-T7-scv2m-replicon, the vector expressing the double mutant RdRp.

2. Transcription of the RdRp Mutant Replicon RNA

RNA was transcribed as described above in Example 1, and linearized with NotI.

3. Electroporation of 293T Cells with the RdRp Mutant Replicon RNA

Cells were electroporated with replicon RNA as described above in Example 1.

4. Detection of Sg mRNA Expressing Luc-GFP Fusion Protein

Sg mRNA expressing Luc-GFP was detected as described above in Example 1.

B. Results

As expected, the mutant replicon RNA failed to express GFP in transfected A549, Calu-1, and Huh-7.5 cells (FIG. 1C). in contrast, all 3 cell lines could fully support the replication of wild type replicons. The data further demonstrated that reporter activities result from active replicon RNA replication and can be used as a readout to evaluate antiviral activity against enzymes encoded by the SARS-CoV-2 replicon.

### Example 3: Dose-Dependent Inhibition of the SARS-CoV-2 Replicon by GS-441524 and GC376

A. Methods

1. Compound Testing in 293T Cells

In order to demonstrate the ability of the T7-scv2-luc-GFP replicon (SEQ ID NO: 2) to respond to specific inhibition, a series of control experiments were performed. The compounds chosen for these initial studies were GS-441524, the parent compound of the remdesivir prodrug, and GC376. Remdesivir, also known as GS-5734, was developed as an anti-viral against Ebola virus (Warren, Travis K et al. “Therapeutic efficacy of the small molecule GS-5734 against Ebola virus in rhesus monkeys.” Nature vol. 531,7594 (2016): 381-5. doi:10.1038/nature17180), specifically targeting RdRp (Gordon, Calvin J et al. “Remdesivir is a direct-acting antiviral that inhibits RNA-dependent RNA polymerase from severe acute respiratory syndrome coronavirus 2 with high potency.” The Journal of biological chemistry vol. 295, 20 (2020): 6785-6797. doi:10.1074/jbc.RA120.013679). Remdesivir is currently the only drug approved by the US Food and Drug Administration (FDA) to treat hospitalized COVID-19 patients (Grein, Jonathan et al. “Compassionate Use of Remdesivir for Patients with Severe Covid-19.” The New England journal of medicine vol. 382, 24 (2020): 2327-2336. doi:10.1056/NEJMoa2007016). Another notable compound GC376, a broad-spectrum inhibitor against 3C or 3C-like proteases of picornaviruses, is also reported to exert a potent inhibition for the Mpro (main protease, also called 3CLpro) encoded by SARS-CoV-2 (Hung, Hui-Chen et al. “Discovery of M Protease Inhibitors Encoded by SARS-CoV-2.” Antimicrobial agents and chemotherapy vol. 64, 9 e00872-20. 20 Aug. 2020, doi:10.1128/AAC.00872-20).

In vitro transcribed and capped replicon RNA was prepared and electroporated into 293T cells as described above in Example 1. After electroporation the 293T cells were incubated with increasing concentrations of GS-441524 or GC376 in a 384 well plate. Replicon inhibition was determined by reduction in the number of GFP positive cells and luciferase activity relative to the DMSO control treated cells. GFP positive cells and luciferase activity were recorded at 30 h after electroporation, by counting the number of green cells in each well using an Acumen® eX3 scanner (sptlabtech) or by measuring luciferase activity using EnVision (PerkinElmer).

2. Quantification and Data Analysis

All numerical data are presented as the mean±SD (standard deviations). The 50% effective concentration, EC50, was calculated determined a non-linear, four-parameter curve fitting method using ActivityBase. Data presented in FIG. 1 was prepared using Prism (GraphPad).

B. Results

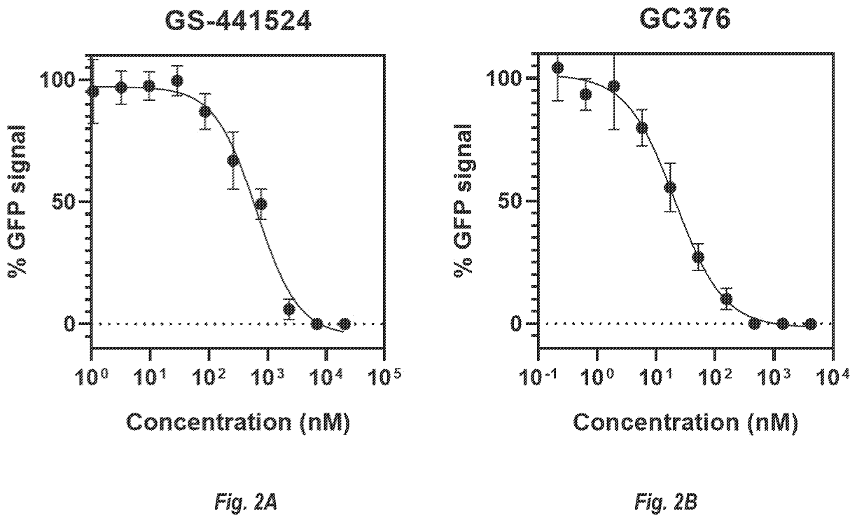

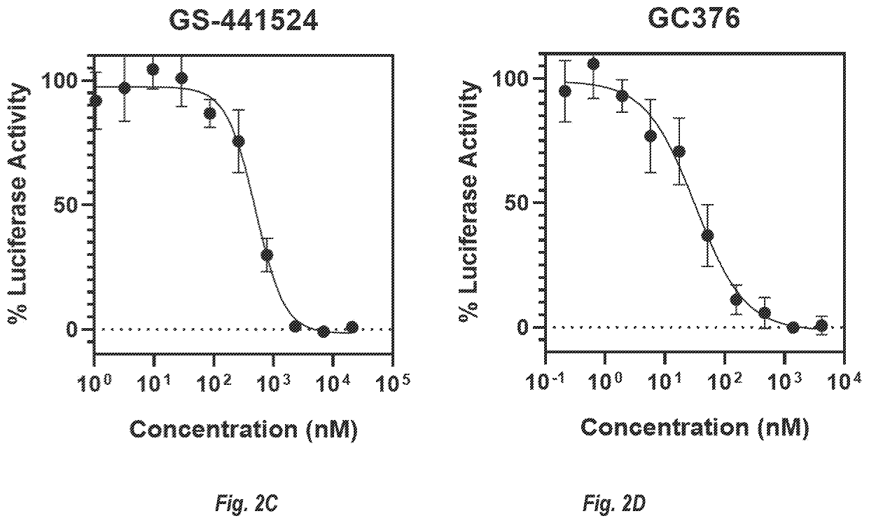

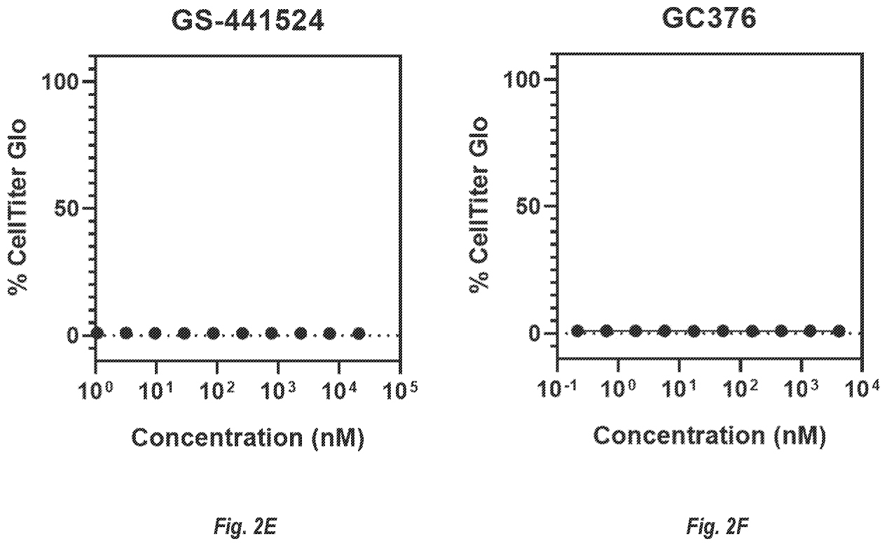

As shown in FIGS. 2A-D, both compounds exhibited antiviral activities in a dose response manner, using either GFP or luciferase as a readout.

The potencies of GS-441524 in inhibiting the SARS-CoV-2 replicon replication, determined by either % GFP signal or % luciferase activity, were 644.58±54.27 nM or 502.49±109.37 nM respectively. The potencies of GC376 in inhibiting the SARS-CoV-2 replicon replication, determined by either % GFP signal or % luciferase activity, were 22.01±5.13 nM or 29.22±9.17 nM respectively. Even though luciferase readout showed higher variabilities, the EC50 values measured by both reporters were comparable in this assay. No cytotoxicity was detected for either compound at the range of concentrations used in this study (FIGS. 2E, F).

### Example 4: Response of the Replicon to a Panel of Compounds that are Specific to SARS-CoV-2

A. Methods

1. SARS-CoV-2 Replicon with a T7 or T7GG Promoter

The T7-scv2-luc-GFP replicon of this example was previously described in Example 1.

A second SARS-CoV-2 replicon of this example, T7GG-scv2-luc-GFP replicon, comprises a T7GG promoter instead of a T7 promoter. The T7GG promoter comprises an extra G residue at the 3′ end of the minimal T7 promoter. It is a variation of the T7 promoter that enhances the yield of in vitro RNA transcription by 5-7 fold. Replicon RNA was prepared from the T7GG-scv2-luc-GFP replicon and electroporated into cells as described above in Example 1.

2. Cell Lines

Cell lines 293T (Clontech, catalog #632180), HEK 293, human lung carcinoma A549 (ATCC®, CCL-185), and human hepatoma Huh7.5 (Apath) cells were maintained in a high-glucose Dulbecco's modified Eagle's medium (DMEM, Invitrogen, catalog #10-013-CV). African green monkey kidney Vero (ATCC, CCL-91) and Vero E6 (ATCC, CRL-1586) cells were cultured in Eagle's Minimum Essential Medium (ATCC®, 30-2003™). Human lung epidermoid carcinoma Calu-1 (ATCC, HTB-54) cells were cultured in McCoy's 5a Medium (ATCC®, 30-2007™). All media were supplemented with 10% fetal bovine serum (FBS; Invitrogen, catalog #10082147) and 1% penicillin/streptomycin. Cells were grown at 37° C. with 5% CO₂.

3. Compound Testing

We tested a panel of published SARS-CoV-2 specific compounds with different reported modes of action (MOA), and sought to determine the impact of various cell lines on the potencies of those compounds. Compound plates were prepared by dispensing compounds (0.2 μl/well) dissolved in DMSO into wells of 384 well poly D lysine-coated plates (Corning 356663) or cell culture-treated, flat-bottom microplate (Corning 3571) using an ECHO acoustic dispenser. Each compound was tested in a 10-point serial 3-fold dilution (final concentrations 42016 nM-2.1 nM). Controls included DMSO only and GS-441524 (final concentration 10 μM). 20,000 transfected cells were added (50 μL/well) using Agilent Bravo to compound plates and the cells were maintained at 37° C./5% CO₂/90% relative humidity.

Percent GFP signal and percent luciferase activity were determined as described above in Example 3.

4. Quantification and Data Analysis

EC50 data were determined as described above in Example 3.

B. Results

In 293T cells, the antiviral potencies of GS-441524 and GC376 measured by T7GG-scv2-luc-GFP replicon (Table 2) were comparable to those determined by T7-scv2-luc-GFP replicon (Table 1, FIGS. 2A-D).

TABLE 1
Antiviral activity against T7-scv2-
luc-GFP replicon (SEQ ID NO: 2)

| | Com- | Mode of | | EC50 |
|---|---|---|---|---|
| Target | pound | action | Cells | (nM) |
| TMPRSS2 | Nafamostat | Entry | 293T | >42016.67 ± 0.0 |
| | | | Vero E6 | >42016.67 ± 0.0 |
| | | | Calu-1 | >42016.67 ± 0.0 |
| | | | A549 | >42016.67 ± 0.0 |
| | Camostat | Entry | 293T | >42016.67 ± 0.0 |
| | | | Vero | >42016.67 ± 0.0 |
| | | | Calu-1 | >42016.67 ± 0.0 |
| | | | A549 | >42016.67 ± 0.0 |
| PIKfyve | Apilimod | Endosome | Calu-1 | >42016.67 ± 0.0 |
| | | entry | Huh-7.5 | >42016.67 ± 0.0 |
| Cathepsin | Relacatib | Endosome | 293T | >42016.67 ± 0.0 |
| L | | entry | Vero | >42016.67 ± 0.0 |
| | | | Calu-1 | >42016.67 ± 0.0 |
| | | | A549 | >42016.67 ± 0.0 |
| RdRp | Remdesivir | RNA | 293T | 11.33 ± 0.0 |
| | | synthesis | Vero | 2238.88 ± 0.0 |
| | | (nucleo- | Huh-7.5 | 18.91 ± 0.0 |
| | | side | Calu-1 | 62.79 ± 0.0 |
| | | prodrug) | A549 | 184.34 ± 0.0 |
| | GS- | RNA | 293T | 602.94 ± 131.37 |
| | 441524 | synthesis | Vero | 302.21 ± 19.82 |
| | | (nucleo- | Huh-7.5 | 1027.09 ± 354.09 |
| | | side) | Calu-1 | 1334.88 ± 269.50 |
| | | | A549 | 1467.59 ± 163.00 |
| Mpro | GC376 | poly- | 293T | 20.82 ± 5.37 |
| | | protein | Vero | 557.39 ± 186.73 |
| | | cleavage | Huh-7.5 | 17.29 ± 5.99 |
| | | | Calu-1 | 52.81 ± 17.38 |
| | | | A549 | 373.17 ± 204.74 |
| | PF- | poly- | 293T | 69.95 ± 14.19 |
| | 00835231 | protein | Vero | 9356.68 ± 0.0 |
| | | cleavage | Huh-7.5 | 175.07 ± 55.95 |
| | | | Calu-1 | 32.74 ± 5.42 |
| | | | A549 | 54.97 ± 0.0 |

TABLE 2
Antiviral activity against T7GG-scv2-luc-GFP replicon

| | Com- | Mode of | | EC50 |
|---|---|---|---|---|
| Target | pound | action | Cells | (nM) |
| TMPRSS2 | Nafamostat | Entry | 293T | >42016.67 ± 0.0 |
| | Camostat | Entry | 293T | >42016.67 ± 0.0 |
| PIKfyve | Apilimod | Endosome | 293T | >42016.67 ± 0.0 |
| | | entry | | |
| Cathepsin | Relacatib | Endosome | 293T | >42016.67 ± 0.0 |
| L | | entry | | |
| RdRp | GS- | RNA | 293T | 656.60 ± 66.40 |
| | 441524 | synthesis | Vero | 324.31 ± 173.44 |
| | | | Huh-7.5 | 830.65 ± 201.37 |
| | | | Calu-1 | 1000.46 ± 217.21 |
| | | | A549 | 715.52 ± 310.70 |
| Mpro | GC376 | poly- | 293T | 50.83 ± 16.37 |
| | | protein | Vero | 900.08 ± 77.11 |
| | | cleavage | Huh-7.5 | 43.94 ± 5.86 |
| | | | Calu-1 | 130.57 ± 21.57 |
| | | | A549 | 1799.80 ± 219.90 |
| | PF- | poly- | 293T | 174.44 ± 53.16 |
| | 00835231 | protein | Vero | 15009.56 ± 3101.34 |
| | | cleavage | Huh-7.5 | 158.66 ± 44.42 |
| | | | Calu-1 | 50.19 ± 4.12 |
| | | | A549 | 128.56 ± 22.37 |
| | Z-LVG | poly- | 293T | >42016.67 ± 0.0 |
| | DMF | protein | Vero | >42016.67 ± 0.0 |
| | | cleavage | Huh-7.5 | >42016.67 ± 0.0 |
| | | | Calu-1 | >42016.67 ± 0.0 |
| | | | A549 | >42016.67 ± 0.0 |

GS-441524 was also active in Vero, Calu-1, A547 and Huh-7.5 cells. GC376 was highly potent in Calu-1 and Huh-7.5 cells, but only modestly active in A549 and Vero cells. In contrast, the EC50 values of PF-00835231 (de Vries, Maren et al. “Comparative study of a 3CLpro inhibitor and remdesivir against both major SARS-CoV-2 clades in human airway models.” bioRxiv preprint doi: doi.org/10.1101/2020.08.28.272880) ranged from 50 to 200 nM in Calu-1, A547, Huh-7.5 cells, and was 75-300 fold lower in Vero cells (EC50 of 15,000 nM). Z-LVG DMF was not active in any of the cells tested. Even though both GC376 and PF-00835231 are protease inhibitors, they showed different cell dependent potency change. The biological mechanism by which the compounds exhibited dramatic differences of antiviral activities in different cells is still unknown. Since the A549, Calu-1 and Huh-7.5 showed similar levels of the transfection efficiencies (FIG. 1C), the shift of potency is unlikely to be caused by GFP expression levels. It is possible the viral Mpro or the compounds are modified or metabolized differently in those cells.

The replicon assay was performed in 384-well plates using automated liquid handling systems, therefore, the format is capable of screening large compound libraries. Results are available as early as 24 h following the drug incubation. The replicon also allows the identification of molecules that target proteins other than RdRp and Mpro whose functions are unknown or for which in vitro enzymatic assays are not available. The replicon also allows identification of any molecules that can affect any other aspects of viral RNA replication. Due to the deletion of the structural proteins, S, E and M, this replicon is not capable of testing compounds that inhibit viral entry, assembly or release.

### Example 5: A Replication Competent, Noninfectious MERS DNA Replicon

A. Methods

1. Construction of the MERS Replicon

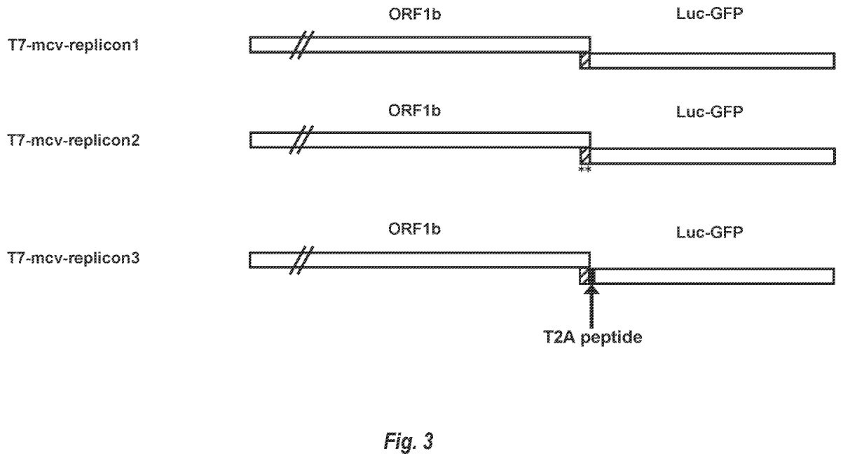

Similar to the SARS-CoV-2 replicon described in Example 1 above, a replicon based on Middle East respiratory syndrome (MERS)-related coronavirus isolate HCoV-EMC/2012 (Genbank: NC_019843.3; SEQ ID NO: 27) was generated. The MERS HcoV-EMC/2012 spike (S) protein open reading frame (ORF) was deleted and replaced with an GFP-firefly luciferase fusion reporter (Luc-GFP). Three different replacements of the S protein with Luc-GFP were created in pSmart BAC vectors. In T7-mcv-replicon1 (SEQ ID NO: 28), Luc-GFP simply replaced the S protein ORF to create a direct fusion with ORF1ab, and a repair insertion was added (see FIG. 3, hatched box). T7-mcv-replicon2 (SEQ ID NO: 29) is similar to T7-mcv-replicon1; however, the S protein start codons were also knocked-out (T21414C, T21498C; see asterisks in FIG. 3), and the S protein start codon was reintroduced after ORF1b. T7-mcv-replicon3 (SEQ ID NO: 30) is also similar to T7-mcv-replicon1; however, a T2A self-cleaving peptide was added after the repair insertion and before Luc-GFP (see diagram in FIG. 3).

The sequences of envelope (E), membrane (M) and their intergenic region were also deleted and replaced with a neomycin resistance gene (Neo). The replicon maintained all the genes and genetic elements necessary for replicating the full-length and subgenomic (sg) RNAs, including replicase complex ORF1ab. Deletion of the structural genes E and M rendered the replicon defective in producing progeny virions. (The N structural gene remains in the replicon). The replicon was linked to a T7 promoter upstream of the 5′ UTR. A cassette containing a 26-nucleotide polyA, a HDV ribozyme and T7 terminator was inserted at the 3′ end of the replicon.

The MERS replicons were assembled as follows. The full-length MERS replicon is −28 k nucleotides long. Five fragments spanning the T7 promoter, full-length MERS, polyA/RbZ/T7 terminator (FIG. 1A), named F1 to F5, with −30 bps overlap were synthesized by Genewiz (South Plainfield, NJ). pSMART BAC vector (Lucigen) was digested with Not I. F1 and F5 fragments were digested with Mlu I. Equimolar amounts of linearized pSMART BAC vector, F1, and F5 were ligated using Gibson Assembly kit (NEB) according to manufacturer's instruction, resulting in pSMART BAC F(1,5). Equimolar amounts of pSMART BAC F(1,5) digested with Aat II and Asc I, and F2 and F4 digested with Mlu I, were ligated using Gibson Assembly kit, resulting in pSMART BAC F(1,2,4,5). Finally, pSMART BAC F(1,2,4,5) digested with Aat II and Asc I, and F3 digested with Mlu I were ligated together using Gibson Assembly Kit, resulting in the full-length noninfectious MERS replicon construct (the pSMART BAC vector with T7-mcv-luc-GFP replicon).

The MERS replicons were then transcribed, and electroporated into 293T cells as described in Example 1 above to test their activity. The numbers of GFP positive cells, as well as GFP intensity were measured.

B. Results

Cells transfected with pSmart BAC T7-mcv-replicon1 (SEQ ID NO: 28, direct fusion of Luc-GFP with ORF1ab) had a detectable but low number of cells expressing GFP and a similarly low intensity of GFP. In contrast, both pSmart BAC T7-mcv-replicon2 (SEQ ID NO: 29, reintroduced start codon) and pSmart BAC T7-mcv-replicon3 (SEQ ID NO: 30, T2A peptide) had a much higher number of cells expressing luc-GFP and similarly high intensity levels of luc-GFP. The qualitative results are summarized in table 3 below.

TABLE 3
Comparison of MERS Replicon Activities

| | Number of GFP- | GFP |
|---|---|---|
| Replicon | positive cells | intensity |
| pSmart BAC T7-mcv- | ++ | ++ |
| replicon1 | | |
| pSmart BAC T7-mcv- | +++++ | +++++ |
| replicon2 | | |
| pSmart BAC T7-mcv- | +++++ | +++++ |
| replicon3 | | |

### Example 6: Response of the MERS Replicon to a Panel of Compounds that are Specific to SARS-CoV-2

A. Methods

Two different SARS coronavirus replicons (T7-scv2-replicon, SEQ ID NO: 13 described above; and T7-scv1-replicon, SEQ ID NO: 32, an earlier SARS virus described below) and a MERS replicon T7-mcv-replicon2 (SEQ ID NO: 29) were tested with a panel of compounds with different MOAs to compare their EC50.

T7-scv1-replicon (Severe acute respiratory syndrome coronavirus 1; SARS-CoV-1) was constructed as described in Example 1 above, using SARS coronavirus isolate CUHK-W1 (GenBank: AY278554; SEQ ID NO: 31) as the basis of the construct. The SARS-CoV-1 spike (S) protein ORF was deleted and replaced with a GFP-firefly luciferase fusion reporter (Luc-GFP). The sequences of envelope (E), membrane (M) and their intergenic region were also deleted and replaced with a neomycin resistance gene (Neo). The full-length SARS-CoV-1 replicon is −28 k nucleotides long. Five fragments spanning the T7 promoter, full-length SARS-CoV-1, polyA/RbZ/T7 terminator (FIG. 1A), named F1 to F5, with −30 bps overlap were synthesized by Genewiz (South Plainfield, NJ). pSMART BAC vector (Lucigen) was digested with Not I. F1 and F5 fragments were digested with Mlu I. Equimolar amounts of linearized pSMART BAC vector, F1, and F5 were ligated using Gibson Assembly kit (NEB) according to manufacturer's instruction, resulting in pSMART BAC F(1,5). Equimolar amounts of pSMART BAC F(1,5) digested with Aat II and Asc I, F2 digested with Mlu I and F4 digested with Pme I, were ligated using Gibson Assembly kit, resulting in pSMART BAC F(1,2,4,5). Finally, pSMART BAC F(1,2,4,5) digested with Aat II and Asc I, and F3 digested with Pme I were ligated together using Gibson Assembly Kit, resulting in the full-length noninfectious SARS-Cov-1 replicon construct (the pSMART BAC vector with T7-scv1-luc-GFP replicon).

293Tcells were transfected with each of the replicons and compounds were tested on the transfected cells as described in Example 4 above.

TABLE 4
Antiviral activity (EC50) against coronavirus replicons
pSmart BAC T7-scv2-replicon, pSmart BAC T7-scv1-replicon,
pSmart BAC T7-mcv-replicon in 293T cells

| | Com- | T7-scv2- | T7-scv1- | T7-mcv- |
|---|---|---|---|---|
| Target | pound | replicon | replicon | replicon |
| RdRp | GS-5734 | 15.6 ± 5.1 | 42.9 ± 3.4 | 81.5 ± 8.8 |
| | (Remdesivir) | (n = 5) | (n = 4) | (n = 4) |
| | GS- | 603 ± 105.5 | 1110 ± 86.5 | 5580 ± 356.9 |
| | 441524 | (n = 22) | (n = 12) | (n = 3) |
| | (Remdesivir | | | |
| | parent) | | | |
| Mpro | GC373 | 19.7 ± 4.6 | 74 ± 4.5 | 29.2 ± 3.7 |
| | | (n = 11) | (n = 10) | (n = 3) |
| | GC376 | 20.8 ± 5.6 | 75.9 ± 23.2 | 35.2 ± 5.3 |
| | | (n = 59) | (n = 12) | (n = 3) |
| | PF- | 74.8 ± 20.0 | 167 ± 15.3 | 101 ± 33.0 |
| | 00835231 | (n = 37) | (n = 14) | (n = 4) |
| | MK-3034 | 3550 ± 346.5 | 6410 ± 427.4 | 4670 ± 518.6 |
| | (Boceprevir) | (n = 2) | (n = 8) | (n = 3) |

Compounds targeting RdRp (GS-5734 and GS-441524) require a greater concentration to achieve 50% inhibition in cells transfected with MERS replicon (T7-mcv-replicon) than in cells transfected with either the SARS-CoV-2 replicon (T7-scv2-replicon) or the SARS-CoV-1 replicon (T7-scv1-replicon). Compounds targeting Mpro have similar EC50 concentrations in cells transfected with MERS replicon (T7-mcv-replicon) compared to cells transfected with SARS-CoV-2 replicon (T7-scv2-replicon).

Table 5 below provides a listing of certain sequences referenced herein. Nucleotide sequences are provided from 5′ to 3′. Amino acid sequences are provided from N-terminus to C-terminus.

Lengthy table referenced here
US20230357870A1-20231109-T00001
Please refer to the end of the specification for access instructions.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

As used herein, the term “about” used in quantitative terms refers to plus or minus 10% of the value it modifies (rounded up to the nearest whole number if the value is not sub-dividable, such as a number of molecules or nucleotides).

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

LENGTHY TABLES
The patent application contains a lengthy table section. A copy of the table is available in electronic form from the USPTO web site (https://seqdata.uspto.gov/?pageRequest=docDetail&DocID=US20230357870A1). An electronic copy of the table will also be available from the USPTO upon request and payment of the fee set forth in 37 CFR 1.19(b)(3).

What is claimed is:

1. A coronavirus reporter replicon comprising:
a. one or more SARS-CoV-2 genes for coronavirus viral RNA replication, wherein the one or more SARS-CoV-2 genes comprise ORF1ab (positions 266-21,555 of SEQ ID NO: 1), ORF3a (positions 25,393-26,220 of SEQ ID NO: 1), ORF6 (positions 27,202-27,387 of SEQ ID NO: 1), ORF7a/b (positions 27,394-27,887 of SEQ ID NO: 1), ORF8 (positions 27,894-28,259 of SEQ ID NO: 1), and ORF10 (positions 29,558-29,674 of SEQ ID NO: 1), or sequences that are at least 90%, 91%, 92%, 93%, 94%, 95%, 96%, 97%, 98%, or 99% homologous to those genes; and
b. at least one reporter gene; and
wherein one or more of a spike ORF, an envelope ORF, and a membrane ORF is replaced with the reporter gene, and
wherein the coronavirus reporter replicon is replication competent.

2.-15. (canceled)

16. The coronavirus reporter replicon of claim 1, wherein at least one of the SARS-CoV-2 genes encodes RNA-dependent RNA polymerase (RdRp) and wherein the RdRp sequence is at least 90% homologous to SEQ ID NO: 9.

17.-23. (canceled)

24. The coronavirus reporter replicon of claim 1, wherein the at least one reporter gene comprises a gene encoding nanoluciferase or a fusion gene encoding firefly luciferase and GFP.

25.-31. (canceled)

32. The coronavirus reporter replicon of claim 1, wherein the coronavirus reporter replicon comprises nucleotides 284-28,130 of SEQ ID NO: 2 or nucleotides 284-26,118 of SEQ ID NO: 5, or
a sequence that is 90%, 91%, 92%, 93%, 94%, 95%, 96%, 97%, 98%, or 99% homologous to nucleotides 284-28,130 of SEQ ID NO: 2 or nucleotides 284-26,118 of SEQ ID NO: 5.

33. The coronavirus reporter replicon of claim 1, wherein the coronavirus reporter replicon comprises nucleotides 284-28,130 of SEQ ID NO: 2 or a sequence that is 90%, 91%, 92%, 93%, 94%, 95%, 96%, 97%, 98%, or 99% homologous to nucleotides 284-28,130 of SEQ ID NO: 2.

34. The coronavirus reporter replicon of claim 1, wherein the coronavirus reporter replicon comprises nucleotides 284-26,118 of SEQ ID NO: 5 or a sequence that is 90%, 91%, 92%, 93%, 94%, 95%, 96%, 97%, 98%, or 99% homologous to nucleotides 284-26,118 of SEQ ID NO: 5.

35.-40. (canceled)

41. A vector comprising the sequence encoding the coronavirus reporter replicon of claim 1.

42. The vector of claim 41, further comprising
a. at least one promoter, wherein the at least one promoter comprises a T7 promoter, a T7GG promoter, a T7GGG promoter, an EF-1a promoter, a human pol I promoter, a CMV promoter, SP6, or a CMV/T7 dual promoter; and
b. a terminator cassette, wherein the terminator cassette comprises a polyA sequence, hepatitis D virus ribozyme sequence, and/or T7 terminator.

43.-61. (canceled)

62. A cell line transfected with the vector of claim 41.

63. (canceled)

64. The cell line of claim 62, wherein the cell line is HEK 293T, Huh7.5, Calu-1, A549, or Vero.

65. (canceled)

66. A method for assaying a candidate agent for inhibition of SARS-CoV-2 replication and/or translation comprising:
a. providing the cell line of claim 62,
b. combining the cell line with a candidate agent for inhibiting SARS-CoV-2; and
c. determining whether the candidate agent decreases the expression and/or activity of the reporter gene.

67.-71. (canceled)

72. A coronavirus reporter replicon comprising:
a. one or more Middle East Respiratory Syndrome (MERS) genes for coronavirus viral RNA replication, wherein the one or more MERS genes comprise ORF1ab (positions 279-21,514 of SEQ ID NO: 27), ORF3 (positions 25,532-25,843 of SEQ ID NO: 27), ORF4a (positions 25,852-26,181 of SEQ ID NO: 27), ORF4b (positions 26,093-26,833 of SEQ ID NO: 27), ORF5 (positions 26,840-27,514 of SEQ ID NO: 27), and ORF8b (positions 28,762-29,100 of SEQ ID NO: 27), or sequences that are at least 90%, 91%, 92%, 93%, 94%, 95%, 96%, 97%, 98%, or 99% homologous to those genes; and
b. at least one reporter gene; and
wherein one or more of a spike ORF, an envelope ORF, and a membrane ORF is replaced with the reporter gene, and
wherein the coronavirus reporter replicon is replication competent.

73. The coronavirus reporter replicon of claim 72, wherein at least one of the MERS genes encodes RNA-dependent RNA polymerase (RdRp) and wherein the RdRp sequence is at least 90% homologous to SEQ ID NO: 34.

74. The coronavirus reporter replicon of claim 72, wherein the at least one reporter gene comprises a gene encoding nanoluciferase or a fusion gene encoding firefly luciferase and GFP.

75. The coronavirus reporter replicon of claim 72, wherein the coronavirus reporter replicon comprises nucleotides 296-28,103 of SEQ ID NO: 28, nucleotides 296-28,104 of SEQ ID NO: 29, or nucleotides 296-28,172 of SEQ ID NO: 30, or
a sequence that is 90%, 91%, 92%, 93%, 94%, 95%, 96%, 97%, 98%, or 99% homologous to nucleotides 296-28,103 of SEQ ID NO: 28, nucleotides 296-28,104 of SEQ ID NO: 29, or nucleotides 296-28,172 of SEQ ID NO: 30.

76. The coronavirus reporter replicon of claim 72, wherein the coronavirus reporter replicon comprises nucleotides 296-28,103 of SEQ ID NO: 28 or a sequence that is 90%, 91%, 92%, 93%, 94%, 95%, 96%, 97%, 98%, or 99% homologous to nucleotides 296-28,103 of SEQ ID NO: 28.

77. The coronavirus reporter replicon of claim 72, wherein the coronavirus reporter replicon comprises nucleotides 296-28,104 of SEQ ID NO: 29 or a sequence that is 90%, 91%, 92%, 93%, 94%, 95%, 96%, 97%, 98%, or 99% homologous to nucleotides 296-28,104 of SEQ ID NO: 29.

78. The coronavirus reporter replicon of claim 72, wherein the coronavirus reporter replicon comprises nucleotides 296-28,172 of SEQ ID NO: 30 or a sequence that is 90%, 91%, 92%, 93%, 94%, 95%, 96%, 97%, 98%, or 99% homologous to nucleotides 296-28,172 of SEQ ID NO: 30.

79. A vector comprising the sequence encoding the coronavirus reporter replicon of claim 72.

80. The vector of claim 79, further comprising
a. at least one promoter, wherein the at least one promoter comprises a T7 promoter, a T7GG promoter, a T7GGG promoter, an EF-1a promoter, a human pol I promoter, a CMV promoter, SP6, or a CMV/T7 dual promoter; and
b. a terminator cassette, wherein the terminator cassette comprises a polyA sequence, hepatitis D virus ribozyme sequence, and/or T7 terminator.

81. A cell line transfected with the vector of claim 79.

82. The cell line of claim 81, wherein the cell line is HEK 293T, Huh7.5, Calu-1, A549, or Vero.

83. A method for assaying a candidate agent for inhibition of MERS replication and/or translation comprising:
a. providing the cell line of claim 81,
b. combining the cell line with a candidate agent for inhibiting MERS; and
c. determining whether the candidate agent decreases the expression and/or activity of the reporter gene.

84. A coronavirus reporter replicon comprising:
a. one or more SARS-CoV-1 genes for coronavirus viral RNA replication, wherein the one or more SARS-CoV-1 genes are chosen from ORF1ab (positions 250-21,470 of SEQ ID NO: 31), ORF3a (positions 25,253-26,077 of SEQ ID NO: 31), ORF6 (positions 27,059-27,250 of SEQ ID NO: 31), ORF7a (positions 27,258-27,626 of SEQ ID NO: 32), ORF7b (positions 27,623-27,757 of SEQ ID NO: 31), ORF8a (positions 27,764-27,883 of SEQ ID NO: 31), and ORF8b (positions 27,849-28,103 of SEQ ID NO: 31) or sequences that are at least 90%, 91%, 92%, 93%, 94%, 95%, 96%, 97%, 98%, or 99% homologous to those genes; and
b. at least one reporter gene; and
wherein one or more of a spike ORF, an envelope ORF, and a membrane ORF is replaced with the reporter gene, and
wherein the coronavirus reporter replicon is replication competent.

85. The coronavirus reporter replicon of claim 84, wherein at least one of the SARS-CoV-1 genes encodes RNA-dependent RNA polymerase (RdRp) and wherein the RdRp sequence is at least 90% homologous to SEQ ID NO: 35.

86. The coronavirus reporter replicon of claim 84, wherein the at least one reporter gene comprises a gene encoding nanoluciferase or a fusion gene encoding firefly luciferase and GFP.

87. The coronavirus reporter replicon of claim 84, wherein the coronavirus reporter replicon comprises nucleotides 283-27,898 of SEQ ID NO: 32 or a sequence that is 90%, 91%, 92%, 93%, 94%, 95%, 96%, 97%, 98%, or 99% homologous to nucleotides 283-27,898 of SEQ ID NO: 32.

88. A vector comprising the sequence encoding the coronavirus reporter replicon of claim 84.

89. The vector of claim 88, further comprising
a. at least one promoter, wherein the at least one promoter comprises a T7 promoter, a T7GG promoter, a T7GGG promoter, an EF-1a promoter, a human pol I promoter, a CMV promoter, SP6, or a CMV/T7 dual promoter; and
b. a terminator cassette, wherein the terminator cassette comprises a polyA sequence, hepatitis D virus ribozyme sequence, and/or T7 terminator.

90. A cell line transfected with the vector of claim 88.

91. The cell line of claim 90, wherein the cell line is HEK 293T, Huh7.5, Calu-1, A549, or Vero.

92. A method for assaying a candidate agent for inhibition of SARS-CoV-1 replication and/or translation comprising:
a. providing the cell line of claim 90,
b. combining the cell line with a candidate agent for inhibiting SARS-CoV-1; and
c. determining whether the candidate agent decreases the expression and/or activity of the reporter gene.
